(19) United States
(12) Patent Application Publication
(10) Pub. No.: US 2023/0320496 A1
(43) Pub. Date: Oct. 12, 2023

(54) INTRAUTERINE ENVIRONMENT SIMULATION SYSTEM

(71) Applicant: Ontogenetics Inc., Penn Valley, CA (US)

(72) Inventors: Edward BUCKLEY, Fairfield, CT (US); Roy REICHWEIN, Penn Valley, CA (US)

(21) Appl. No.: 18/182,187

(22) Filed: Mar. 10, 2023

Related U.S. Application Data
(63) Continuation-in-part of application No. PCT/US2021/049963, filed on Sep. 10, 2021.
(60) Provisional application No. 63/076,558, filed on Sep. 10, 2020.

(51) Int. Cl. A47D 9/02; G06N 20/00

(52) U.S. Cl. CPC A47D 9/057; G06N 20/00

(57) ABSTRACT

An intrauterine simulation system comprising a motion-control system and/or a maternal heartbeat simulator. The motion-control system may be configured to move a platform supporting an infant in a pattern characteristic of the movement of a woman in a late stage of pregnancy, thereby simulating movement experienced by a fetus in the intrauterine environment. The sound and vibration may be customized to the mother's biometric data to more closely simulate the infant's experience in the womb. The platform supporting the infant may be incorporated into a bassinet, cradle, mattress, and/or other suitable device. A system controller may be configured to gradually reduce aspects of the simulation, such as the intensity of the sound waves and/or vibrations or extent of the movement, thereby transitioning an infant to the extrauterine environment.

### CROSS-REFERENCES

The following applications and materials are incorporated herein, in their entireties, for all purposes: U.S. Provisional Patent Application Ser. No. 63/076,558, filed Sep. 10, 2020; and International Application Serial No. PCT/US2021/049963, filed Sep. 10, 2021.

### FIELD

This disclosure relates to systems and methods for infant care. More specifically, the disclosed embodiments relate to infant bassinets, cradles, mattresses, pods, and the like, configured to at least partially simulate aspects of an intrauterine environment and to transition from that environment to the outside world.

### INTRODUCTION

Perhaps the most difficult transition a mammal is required to make in its lifetime is the change from the intrauterine environment to the extrauterine environment at birth. Every parameter of the infant's environment changes abruptly. Dramatic shifts in temperature, tactile sensation, audio stimuli, motion, and light are exacerbated by conditions in the hospital delivery room where most people in modern societies give birth. Even the environment in a loving home is alarmingly unfamiliar, and many infants exhibit prolonged crying and sleeplessness which may be related to transitional stress. It is believed that these abrupt changes in the environment may tend to intensify the infant's intrauterine to extrauterine transition and may inflict harm which affects the person's emotional and physical response to adaptive or environmental change throughout life. In contrast, in pre-modern times infants continued to be close to their mothers and felt the mother's movements and heartbeat. A natural transition occurred as the baby grew and was carried by the mother less and less. Therefore, a gradual and effective transition of the infant from the intrauterine environment to the extrauterine environment may have substantial long-term as well as short-term benefits.

The transition from an intrauterine environment to an extrauterine environment can possibly be made easier by the aid of a transition system. An effective transition system would duplicate one or more aspects of the intrauterine conditions perceived by the infant just prior to birth. It would also provide means for gradually altering environmental stimuli over time until they reflect the natural extrauterine environment. Known transition systems fail to accurately simulate certain intrauterine conditions, such as the sensation of the mother's heartbeat, the motion characteristic of the mother's gait late in pregnancy, or activity levels relative to the time of day. Additionally, some known transition systems have proven not to be safe for infant sleeping and pose a health risk. Better solutions are needed for simulating the intrauterine environment.

### SUMMARY

The present disclosure provides systems, apparatuses, and methods relating to intrauterine simulation systems.

In some examples, a baby bed of the present disclosure includes: a platform configured to support an infant; a transducer coupled to the platform; a mechanical actuator coupled to the platform; and an electronic controller configured to simulate an intrauterine environment for the infant by simulating a heartbeat using the transducer and simulating a walking gait using the mechanical actuator; wherein one or more settings of the simulated intrauterine environment are determined by a machine learning algorithm based on biometric information of the mother of the infant.

In some examples, a baby bed of the present disclosure includes: a platform configured to support an infant; a transducer coupled to the platform; a mechanical actuator coupled to the platform; and an electronic controller configured to simulate an intrauterine environment for the infant by simulating a heartbeat using the transducer and simulating a walking gait using the mechanical actuator; wherein one or more settings of the simulated intrauterine environment are determined based on female biometric information.

In some examples, a method for transitioning an infant after birth using a simulated intrauterine environment of the present disclosure includes: providing a simulated intrauterine environment by simulating a heartbeat using a transducer coupled to a platform configured to support an infant, and simulating a walking gait by moving the platform using a mechanical actuator; and determining and automatically setting one or more parameters of the simulated intrauterine environment based on biometric information of one or more mothers.

Features, functions, and advantages may be achieved independently in various embodiments of the present disclosure, or may be combined in yet other embodiments, further details of which can be seen with reference to the following description and drawings.

### BRIEF DESCRIPTION OF THE DRAWINGS

FIG. 1 is a set of graphs of illustrative pelvic displacements associated with a woman walking during a third trimester of pregnancy.

FIG. 2 is a schematic diagram of an illustrative intrauterine simulation system, in accordance with aspects of the present teachings.

FIG. 3 is an isometric view of an illustrative bassinet incorporating an intrauterine simulation system, in accordance with aspects of the present teachings.

FIG. 4 is an exploded view of the bassinet of FIG. 3.

FIG. 5 is an isometric view of a mounting structure of the bassinet of FIG. 3 in a collapsed configuration.

FIG. 6 is a side view of a portion of the bassinet of FIG. 3.

FIG. 7 is a plan view of an illustrative motion-control assembly suitable for use with the bassinet of FIG. 3.

FIG. 8 is a side view of an illustrative intrauterine simulation assembly including the motion-control assembly of FIG. 7.

FIG. 9 is magnified view of a portion of the simulation assembly of FIG. 8.

FIG. 10 is magnified side view of an end portion of the simulation assembly of FIG. 8.

FIG. 11 is a schematic diagram depicting an illustrative control system for the simulation assembly of FIG. 7.

FIG. 12 is a schematic top view of another illustrative simulation assembly of the present teachings.

FIG. 13 is a side view of the simulation assembly of FIG. 12.

FIG. 14 is a magnified view of a portion of the simulation assembly of FIG. 12, showing an alternative actuator.

FIG. 15 is a schematic view depicting a dispersion pattern of sound waves produced by an illustrative transducer, in accordance with aspects of the present teachings.

FIG. 16 is a sectional view of an illustrative cushion device including an intrauterine simulation assembly, in accordance with aspects of the present teachings.

FIG. 17 is a schematic diagram of a wearable infant monitor suitable for use with systems of the present disclosure.

FIG. 18 is an illustrative wearable article of clothing incorporating the infant monitor of FIG. 17.

FIG. 19 is a schematic diagram of an illustrative intrauterine simulation system in accordance with aspects of the present disclosure.

FIG. 20 is a schematic diagram depicting an illustrative system for machine learning training and operation.

FIG. 21 is a schematic diagram of an illustrative system for analyzing biometric data in accordance with aspects of the present disclosure.

FIG. 22 is a flowchart depicting steps of an illustrative method for capturing and analyzing pre-partum biometric data in accordance with aspects of the present disclosure.

FIG. 23 is a flowchart depicting steps of an illustrative method for capturing and analyzing postpartum biometric data in accordance with aspects of the present disclosure.

FIG. 24 is a flowchart depicting steps of an illustrative method for transferring customization parameters to a cradle system of the present disclosure.

### DETAILED DESCRIPTION

Various aspects and examples of an intrauterine simulation system including motion-control and intrauterine heartbeat-simulation assemblies, as well as related methods, are described below and illustrated in the associated drawings. Unless otherwise specified, an intrauterine simulation system in accordance with the present teachings, and/or its various components, may contain at least one of the structures, components, functionalities, and/or variations described, illustrated, and/or incorporated herein. Furthermore, unless specifically excluded, the process steps, structures, components, functionalities, and/or variations described, illustrated, and/or incorporated herein in connection with the present teachings may be included in other similar devices and methods, including being interchangeable between disclosed embodiments. The following description of various examples is merely illustrative in nature and is in no way intended to limit the disclosure, its application, or uses. Additionally, the advantages provided by the examples and embodiments described below are illustrative in nature and not all examples and embodiments provide the same advantages or the same degree of advantages.

This Detailed Description includes the following sections, which follow immediately below: (1) Definitions; (2) Overview; (3) Examples, Components, and Alternatives; (4) Advantages, Features, and Benefits; and (5) Conclusion. The Examples, Components, and Alternatives section is further divided into subsections, each of which is labeled accordingly.

### Definitions

The following definitions apply herein, unless otherwise indicated.

“Comprising,” “including,” and “having” (and conjugations thereof) are used interchangeably to mean including but not necessarily limited to, and are open-ended terms not intended to exclude additional, unrecited elements or method steps.

Terms such as “first”, “second”, and “third” are used to distinguish or identify various members of a group, or the like, and are not intended to show serial or numerical limitation.

“AKA” means “also known as,” and may be used to indicate an alternative or corresponding term for a given element or elements.

“Elongate” or “elongated” refers to an object or aperture that has a length greater than its own width, although the width need not be uniform. For example, an elongate slot may be elliptical or stadium-shaped, and an elongate candlestick may have a height greater than its tapering diameter. As a negative example, a circular aperture would not be considered an elongate aperture.

“Coupled” means connected, either permanently or releasably, whether directly or indirectly through intervening components.

“Resilient” describes a material or structure configured to respond to normal operating loads (e.g., when compressed) by deforming elastically and returning to an original shape or position when unloaded.

“Rigid” describes a material or structure configured to be stiff, non-deformable, or substantially lacking in flexibility under normal operating conditions.

“Elastic” describes a material or structure configured to spontaneously resume its former shape after being stretched or expanded.

“Processing logic” describes any suitable device(s) or hardware configured to process data by performing one or more logical and/or arithmetic operations (e.g., executing coded instructions). For example, processing logic may include one or more processors (e.g., central processing units (CPUs) and/or graphics processing units (GPUs)), microprocessors, clusters of processing cores, FPGAs (field-programmable gate arrays), artificial intelligence (AI) accelerators, digital signal processors (DSPs), and/or any other suitable combination of logic hardware.

A “controller” or “electronic controller” includes processing logic programmed with instructions to carry out a controlling function with respect to a control element. For example, an electronic controller may be configured to receive an input signal, compare the input signal to a selected control value or setpoint value, and determine an output signal to a control element (e.g., a motor or actuator) to provide corrective action based on the comparison. In another example, an electronic controller may be configured to interface between a host device (e.g., a desktop computer, a mainframe, etc.) and a peripheral device (e.g., a memory device, an input/output device, etc.) to control and/or monitor input and output signals to and from the peripheral device.

Directional terms such as “up,” “down,” “vertical,” “horizontal,” and the like should be understood in the context of the particular object in question. For example, an object may be oriented around defined X, Y, and Z axes. In those examples, the X-Y plane will define horizontal, with up being defined as the positive Z direction and down being defined as the negative Z direction.

“Providing,” in the context of a method, may include receiving, obtaining, purchasing, manufacturing, generating, processing, preprocessing, and/or the like, such that the object or material provided is in a state and configuration for other steps to be carried out.

In this disclosure, one or more publications, patents, and/or patent applications may be incorporated by reference. However, such material is only incorporated to the extent that no conflict exists between the incorporated material and the statements and drawings set forth herein. In the event of any such conflict, including any conflict in terminology, the present disclosure is controlling.

### Overview

In general, intrauterine simulation systems of the present teachings may include a motion-control assembly and/or a maternal heartbeat simulator. Settings of the system may be determined at least in part by analysis of the mother's prepartum and/or postpartum biometric data (e.g., using wearable sensors and one or more machine learning models). In some examples, the intrauterine simulation system is incorporated into a bassinet, cradle, mattress, cushion, carrier, bed, incubator, and/or other device suitable for supporting an infant. Intrauterine environments may include customized motions and sounds generated from the biometrics of the infant's birth mother.

Known infant environmental transition systems control the generation of motion and sound in a very general way, using only assumed biometric data from an average or nominal “mother.” In other words, all the characteristics of motion and sound were based on averages or standards: pace, heart rate, activity level, duration, time of day, sleep states, etc. No attempt was made to customize the motion and sound of the system to the actual mother's physiological profile and biometric patterns. This leads to a potential increased mismatch between the simulated environment and the infant's actual intrauterine experience.

Systems of the present disclosure overcome these deficiencies by capturing mother's prepartum and/or postpartum biometric data and analyzing the data to generate new control signals for motion and sound generation to approximate as exactly as possible the mother's intrauterine environment as experienced by the infant in the womb.

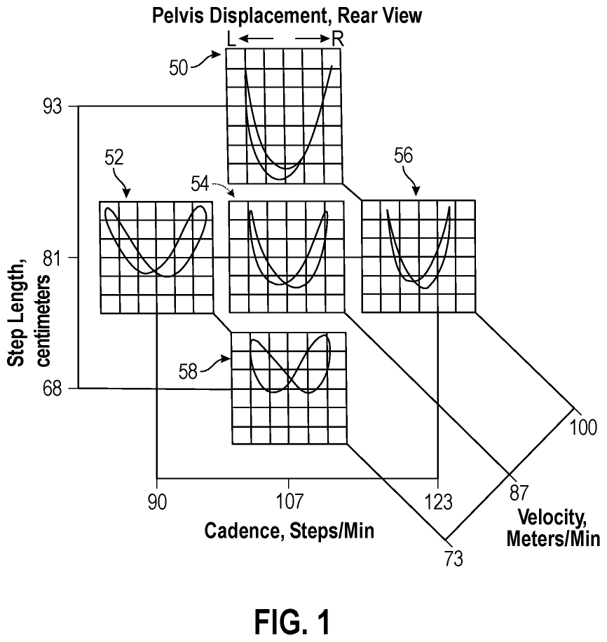

A motion-control assembly in accordance with aspects of the present teachings is configured to simulate aspects of the pre-birth environment for a baby in a cradle, bassinet, or the like. The motion-control assembly may include, e.g., a motorized system coupled to a platform and configured to impart to the platform linear and/or rotational movement suitable for approximating movement experienced by the baby as his or her mother walked. FIG. 1 depicts characteristic displacement patterns of a pelvis of a typical pregnant woman while walking during the third trimester of pregnancy. FIG. 1 depicts a plurality of representative displacement patterns 50, 52, 54, 56, 58 from a rear view of the pelvis. The displacement occurs in both a lateral direction (e.g., left or right) and a vertical direction (up or down). As FIG. 1 shows, the displacement pattern is variable based on factors including the woman's cadence, step length, and velocity, but generally comprises a modified figure-eight pattern. The motion-control assembly of the present teachings is configured to approximate at least some aspects of this pattern, thereby simulating the movement of the intrauterine environment. In some embodiments, the linear and rotational components of the motion-control assembly are modified to replicate the step length and pelvis displacement of the infant's own mother.

A personalized intrauterine environment simulation system in accordance with aspects of the present teachings may include any suitable system configured to approximate as closely as possible the sensations experienced by a fetus due to the intrauterine soundscape (e.g., including heartbeat) and movements of its mother. Unless otherwise specified, the term “intrauterine soundscape” as used herein refers generally to the activity of the maternal heart, internal blood flow, intestinal peristalsis (bowel sounds), and the like and does not refer specifically to any particular cardiac contraction, relaxation, or other aspect of the cardiac cycle or other internal maternal system. In a similar fashion, the term “movement” as used herein refers generally to the movement of the mother, and does not refer specifically to any particular type of movement such as walking, running, climbing stairs, etc.

In general, a fetus in the womb experiences the mother's heartbeat continuously throughout the day and night, and experiences the mother's movements as they occur. These movements may have some type of pattern or periodicity. Short-term patterns may include walking and stair climbing while longer-term patterns may include daily activities such as sleeping, eating, and exercise.

In some examples, the motion-control and/or heartbeat-simulation assemblies of the present disclosure are controlled by an electronic controller (e.g., incorporating processing logic). For example, the controller may drive one or more transducer(s) of the heartbeat-simulation system and/or motor(s) of the motion-control system.

In some examples, the controller is configured to vary certain aspects of the motion-control and/or heartbeat-simulation systems as a function of time. For example, the controller may be configured to sense a time of day (e.g., morning, afternoon, evening, night, etc.) and to vary the simulated motion and heartbeat sensations based on the sensed time. This may increase the accuracy of the intrauterine simulation. For example, the controller may reduce the amplitude of motions created by the motion-control system at night compared to the amplitude of motions during the day. This would be consistent with the intrauterine experience of a fetus gestated by a woman who typically sleeps at night and is awake during the day. Similarly, the pace of a simulated heartbeat may be slower during the night than during the day, and/or may simulate a REM cycle of a sleeping woman. In some examples, the controller may be switchable from a night setting to a day setting based on user input, rather than automatically based on a sensed time.

Additionally, or alternatively, the controller may be configured to gradually reduce aspects of the intrauterine simulation (e.g., amplitude of motion and/or intensity of simulated intrauterine soundscape) on a scale of weeks or months. This may help to gradually acclimate the infant to the extrauterine environment.

In some examples, one or more settings and/or functions of the controller may be modified using a remote control, a software application (e.g., a smartphone app), and/or the like.

Aspects of intrauterine simulation systems of the present disclosure may be embodied as a computer method, computer system, or computer program product. Accordingly, aspects of the intrauterine simulation system may take the form of an entirely hardware embodiment, an entirely software embodiment (including firmware, resident software, micro-code, and the like), or an embodiment combining software and hardware aspects, all of which may generally be referred to herein as a “circuit,” “module,” or “system.” Furthermore, aspects of the intrauterine simulation system may take the form of a computer program product embodied in a computer-readable medium (or media) having computer-readable program code/instructions embodied thereon.

Any combination of computer-readable media may be utilized. Computer-readable media can be a computer-readable signal medium and/or a computer-readable storage medium. A computer-readable storage medium may include an electronic, magnetic, optical, electromagnetic, infrared, and/or semiconductor system, apparatus, or device, or any suitable combination of these. More specific examples of a computer-readable storage medium may include the following: an electrical connection having one or more wires, a portable computer diskette, a hard disk, a random access memory (RAM), a read-only memory (ROM), an erasable programmable read-only memory (EPROM or Flash memory), an optical fiber, a portable compact disc read-only memory (CD-ROM), an optical storage device, a magnetic storage device, and/or any suitable combination of these and/or the like. In the context of this disclosure, a computer-readable storage medium may include any suitable non-transitory, tangible medium that can contain or store a program for use by or in connection with an instruction execution system, apparatus, or device.

A computer-readable signal medium may include a propagated data signal with computer-readable program code embodied therein, for example, in baseband or as part of a carrier wave. Such a propagated signal may take any of a variety of forms, including, but not limited to, electro-magnetic, optical, and/or any suitable combination thereof. A computer-readable signal medium may include any computer-readable medium that is not a computer-readable storage medium and that is capable of communicating, propagating, or transporting a program for use by or in connection with an instruction execution system, apparatus, or device.

Program code embodied on a computer-readable medium may be transmitted using any appropriate medium, including but not limited to wireless, wireline, optical fiber cable, RF, and/or the like, and/or any suitable combination of these.

Computer program code for carrying out operations for aspects of intrauterine simulation system may be written in one or any combination of programming languages, including an object-oriented programming language (such as Java, C++), conventional procedural programming languages (such as C), and functional programming languages (such as Haskell). Mobile apps may be developed using any suitable language, including those previously mentioned, as well as Objective-C, Swift, C #, HTML5, and the like. The program code may execute entirely on a user's computer, partly on the user's computer, as a stand-alone software package, partly on the user's computer and partly on a remote computer, or entirely on the remote computer or server. In the latter scenario, the remote computer may be connected to the user's computer through any type of network, including a local area network (LAN) or a wide area network (WAN), and/or the connection may be made to an external computer (for example, through the Internet using an Internet Service Provider).

Aspects of the intrauterine simulation system may be described below with reference to flowchart illustrations and/or block diagrams of methods, apparatuses, systems, and/or computer program products. Each block and/or combination of blocks in a flowchart and/or block diagram may be implemented by computer program instructions. The computer program instructions may be programmed into or otherwise provided to processing logic (e.g., a processor of a general purpose computer, special purpose computer, field programmable gate array (FPGA), or other programmable data processing apparatus) to produce a machine, such that the (e.g., machine-readable) instructions, which execute via the processing logic, create means for implementing the functions/acts specified in the flowchart and/or block diagram block(s).

Additionally or alternatively, these computer program instructions may be stored in a computer-readable medium that can direct processing logic and/or any other suitable device to function in a particular manner, such that the instructions stored in the computer-readable medium produce an article of manufacture including instructions which implement the function/act specified in the flowchart and/or block diagram block(s).

The computer program instructions can also be loaded onto processing logic and/or any other suitable device to cause a series of operational steps to be performed on the device to produce a computer-implemented process such that the executed instructions provide processes for implementing the functions/acts specified in the flowchart and/or block diagram block(s).

Any flowchart and/or block diagram in the drawings is intended to illustrate the architecture, functionality, and/or operation of possible implementations of systems, methods, and computer program products according to aspects of the intrauterine simulation system. In this regard, each block may represent a module, segment, or portion of code, which comprises one or more executable instructions for implementing the specified logical function(s). In some implementations, the functions noted in the block may occur out of the order noted in the drawings. For example, two blocks shown in succession may, in fact, be executed substantially concurrently, or the blocks may sometimes be executed in the reverse order, depending upon the functionality involved. Each block and/or combination of blocks may be implemented by special purpose hardware-based systems (or combinations of special purpose hardware and computer instructions) that perform the specified functions or acts.

Unless indicated otherwise, a “mother” may include any birthing person or birthing mammal. While the overview above and the examples below focus upon simulating an intrauterine environment for a human infant, the present teachings also may be used for other mammal species. For example, puppies and kittens may benefit from a simulated intrauterine environment. In that case, systems according to the present teachings might include alternative (and in some examples, more complex) motions, sounds, and/or vibrations, based on simulating four-legged motions of the mother as well as sounds from multiple other siblings in the womb. To some extent, such motions and sounds would also occur outside the womb after the litter is born. Similarly, the present teachings can be used to simulate the intrauterine and/or natural infant environment of any animal species, with beneficial uses for pet owners, breeders, veterinarian clients, and/or zoos, among others.

Furthermore, the present teachings extend to multiple human births (e.g., twins, triplets, and so forth), in which case systems according to the present teachings may be configured to simulate the resulting more complicated intrauterine environments. For example, the present teachings may include systems that accommodate more than one human infant, and/or that simulate the additional intrauterine movements, sounds, and vibrations associated with a multiple pregnancy.

### Examples, Components, and Alternatives

The following sections describe selected aspects of illustrative intrauterine simulation systems as well as related systems and/or methods. The examples in these sections are intended for illustration and should not be interpreted as limiting the scope of the present disclosure. Each section may include one or more distinct embodiments or examples, and/or contextual or related information, function, and/or structure.

A. Illustrative Intrauterine Simulation System

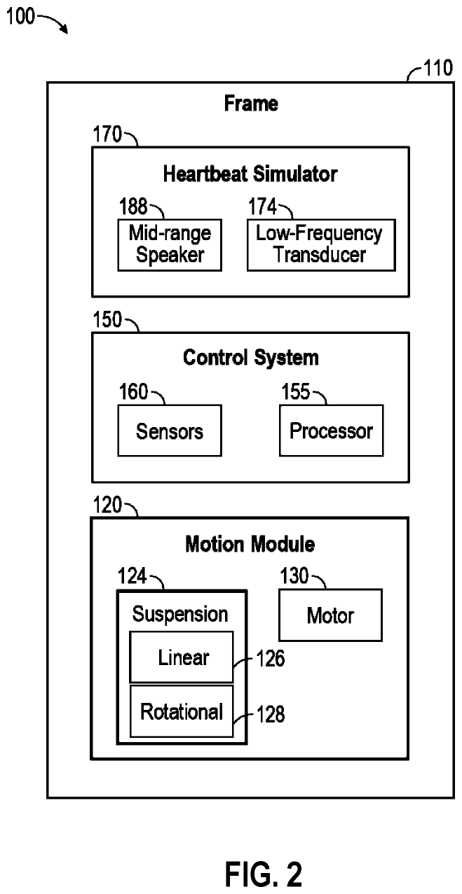

With reference to FIG. 2, this section describes an illustrative intrauterine simulation system 100, which is an example of the intrauterine simulation systems described above.

System 100 includes a frame 110 having a platform or expanse configured to support an infant (directly or indirectly). For example, frame 110 may include a thin, substantially rigid platform configured to support a bed and/or bedding for an infant. In some examples, the platform comprises a mesh fabric pulled taut within a sturdy outer frame. Frame 110 may further include a portion forming a baby bed, such as a bassinet, cradle, or other suitable structure, to which the platform is movably attached (e.g., such that the platform can be rotated and/or translated relative to the bassinet structure).

System 100 further includes a motion module 120, which is an example of a motion-control assembly. Motion module 120 may comprise any suitable mechanical components, such as a mechanical actuator (e.g., a linear actuator and/or a rotational actuator), configured to impart movement to the movable platform of frame 110. For example, motion module 120 may include a suspension and drive system 124 having a linear-motion actuator 126 (AKA a linear actuator) configured to linearly displace the movable platform relative to the fixed frame, and a rotational-motion actuator 128 (AKA a rotational actuator) configured to rotate the movable platform relative to the fixed frame. In some examples, linear-motion actuator 126 and rotational-motion actuator 128 are coupled together (e.g., driven by a common motor and/or common belts and pulleys) to produce the complex figure-eight motion characteristic of third-trimester pelvic movement while walking. In other examples, the linear and rotational actuators are decoupled from each other to produce the figure-eight motion or can be actuated independently or together in various combinations to produce a multitude of patterns. In some examples, multiple motors may be used to separately drive the linear and rotational movements.

Suspension and drive system 124 may include any suitable assembly of linear and/or rotary actuators, belts and pulleys, chains and gears, and/or any other suitable devices.

Motion module 120 further includes a motor 130 configured to drive suspension and drive system 124. In some examples, motor 130 comprises a DC motor (brushed or brushless), such as a permanent-magnet type motor. Alternatively, or additionally, motor 130 may comprise a shunt motor, stepper motor, compound motor, and/or series motor. In some examples, motor 130 comprises an AC motor. Typically, motor 130 is suitable for quiet and smooth operation, so that operation of the motor does not disturb an infant sleeping in system 100.

System 100 further includes a controller or control system 150. Control system 150 has processing logic including one or more processors 155, which may comprise a computer, microprocessor, and/or any other suitable device. Processors 155 may be configured to control motor 130 (e.g., a state and/or speed of the motor). Additionally, or alternatively, processors 155 may be configured to control one or more transducers, described below.

Control system 150 may include and/or be in communication with a plurality of sensors 160. In some examples, sensors 160 include sensors configured to obtain data facilitating control of motor 130 by processor 155. For example, sensors 160 may include one or more encoders and/or speed sensors attached to motor 130, which may be used in a feedback algorithm implemented by processor 155 to maintain a speed of the motor at a target speed. Sensors 160 may further include sensors configured to obtain safety-related information, such as proximity sensors (e.g., for monitoring platform movement and position), current sensors (e.g., for identifying excessive current in the event of a system malfunction), temperature sensors (e.g., for identifying overheating malfunctioning components), load cells (e.g., for detecting an unexpected weight distribution on frame 110), microphones (e.g., for detecting an infant's cry), and/or the like. Control system 150 may further be configured to display and/or communicate information related to the sensed information. For example, control system 150 may include one or more visible and/or audible alarms configured to be activated in response to detection of information indicating a malfunction. Additionally, or alternatively, an alert may be transmitted to an external device (e.g., a smartphone, a dedicated receiver, etc.).

System 100 further includes a heartbeat simulator 170, which is an example of a system configured to simulate sensations of a maternal heartbeat as experienced in the intrauterine environment, as described above. Heartbeat simulator 170 includes a transducer 174, which may be a low-frequency transducer. Transducer 174 is configured to produce suitable sound waves and/or vibrations. In some examples, the transducer may comprise a bass transducer, bass shaker, haptic motor or engine, tactile transducer, and/or any other suitable device(s) configured to produce a low-frequency waveform suitable for simulating the maternal intrauterine heartbeat. In some examples, transducer 174 is configured to produce low-frequency sound waves and/or vibrations (e.g., having a fundamental frequency less than approximately 50 Hz, a majority of energy in the range of approximately 20 to 80 Hz, and/or any other suitable low-frequency sound waves and vibrations).

Optionally, heartbeat simulator 170 further includes a standard mid-range transducer 188. Mid-range transducer 188 may be configured to produce higher-frequency sound waves (e.g., predominantly above approximately 100 Hz, in a range of approximately 100 Hz to 10,000 Hz, and/or any other suitable mid-frequency range). These higher-frequency sound waves may augment the low-frequency waves produced by transducer 174 in order to produce a desired spectrum of sound and/or vibrational waves. Alternatively, or additionally, transducer 188 may be coupled to a smartphone or other communications device to play music, a human voice, an alarm, and/or any other suitable sound.

Transducer 174 and/or transducer 188 are controlled by control system 150, which may include one or more amplifiers coupled to the transducers.

Processor 155 and/or any other suitable component of control system 150 may be configured to receive data from and/or send data to a remote control, smart phone, personal computer, laptop, or other suitable device. Data received by the controller may comprise instructions to be implemented by the controller for controlling motion module 120, heartbeat simulator 170, and/or any other suitable component of system 100. For example, processor 155 may be configured to receive instructions relating to a target speed of motor 130, a volume and/or frequency of transducer 174, etc. Data sent by the controller may include current operating state, sensor data, and/or other information related to the status or performance of the system.

B. Illustrative Bassinet

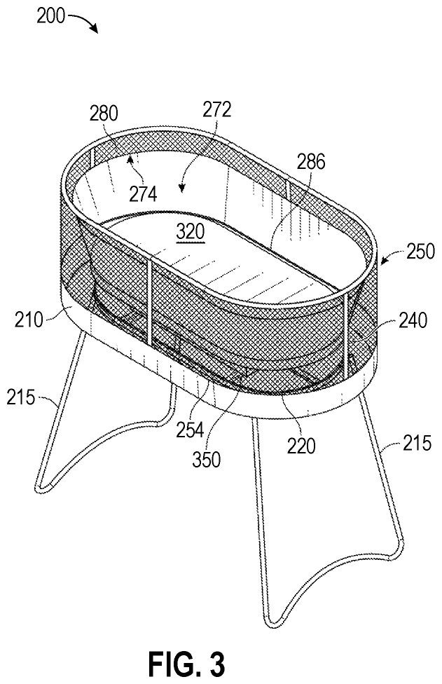

As shown in FIGS. 3-6, this section describes an illustrative bassinet 200 or baby bed in accordance with aspects of the present teachings. Bassinet 200 is an example of a system configured to simulate an intrauterine environment, as described above.

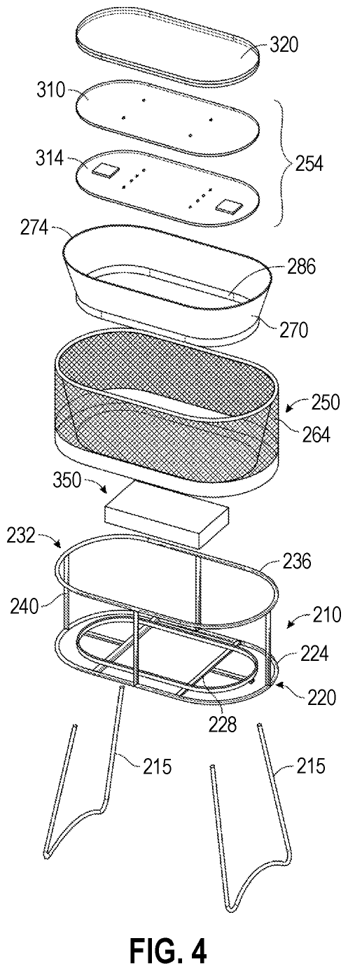

FIG. 3 is an isometric view of bassinet 200, and FIG. 4 is an exploded view of the bassinet. As depicted in FIGS. 3-4, bassinet 200 includes a base 210 supported by legs 215. In the depicted example, base 210 is supported above the ground by a pair of legs 215, but in other examples, the base may be supported by any number of legs and/or other suitable structure. Legs 215 may be adjustable to support base 210 at a selectable height above the ground. In some cases, the legs also may be independently adjustable, to allow the bassinet to be angled to a desired degree. For example, this may allow an infant to recline with its head slightly elevated, to address infant reflux.

Base 210 has a horizontal portion 220, which includes a base rim 224 extending around a perimeter of the horizontal portion and a plurality of base bars 228 supported by the base rim. In other examples, horizontal portion 220 may comprise a single planar structure rather than a rim and a plurality of base bars. However, the rim and base bars may provide improved air flow relative to a single plane. Additionally, or alternatively, the rim and base bars may attenuate low-frequency vibrations less than a planar base would.

Base 210 further includes a mounting structure 232 extending substantially transversely from horizontal portion 220. Mounting structure 232 has an upper rim 236 supported above base 210 by a plurality of support columns 240. Upper rim 236 is configured to support a mesh framework 250, and the mesh framework is configured to support a platform 254 above horizontal base portion 220.

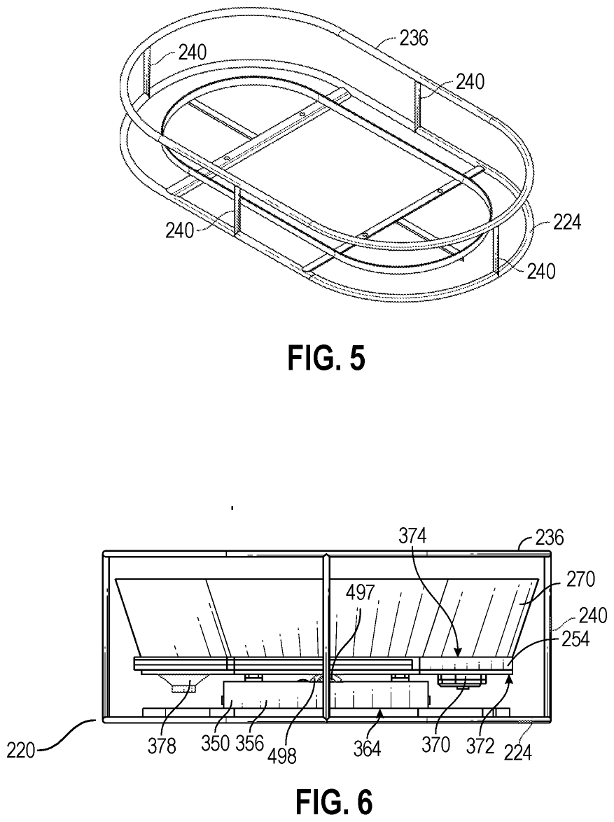

As shown in FIG. 5, support columns 240 may be collapsible, allowing base 210 to be transitioned to a more compact collapsed configuration. The collapsed configuration may be convenient for storing and/or transporting the bassinet. Additionally, or alternatively, the bassinet may be used in the collapsed configuration with an infant in the bassinet. This may be convenient because the sides of the bassinet are shorter in the collapsed configuration, such that an infant inside the bassinet may be easier to reach. The shortened sides of the bassinet may have a height sufficient to satisfy regulatory safety requirements, such as the requirements imposed by the U.S. Consumer Product Safety Commission.

Returning to FIGS. 3-4, mesh framework 250 may be configured to support platform 254 above the motion-control module in any suitable manner. In the depicted example, mesh framework 250 includes a substantially rigid outer wall 264 coupled to an elastic inner wall 270. Outer wall 264 is coupled to base rim 224 and to upper rim 236 of base 210 and forms a wall extending between the base rim and the upper rim. A partially enclosed interior 272 is defined by outer wall 264. In the depicted example, outer wall 264 comprises a substantially rigid mesh fabric, but in other examples the outer wall may comprise any other suitable material or may be omitted.

Elastic inner wall 270 is disposed within interior 272 and may be coupled to outer wall 264 in any suitable manner. In the depicted example, an upper edge 274 of elastic inner wall 270 is rigidly connected (e.g., via stitches, adhesive, and/or any other suitable fasteners) to an inner portion 280 of outer wall 264 which extends into interior 272. Accordingly, elastic inner wall 270 is suspended from upper rim 236 by inner portion 280 of outer wall 264. In other examples, elastic inner wall 270 may be coupled to outer wall 264 and/or to base 210 in any other suitable way. In some examples, elastic inner wall 270 and outer wall 264 are formed by a same piece of fabric.

A lower end 286 of elastic inner wall 270 is rigidly attached to platform 254 (e.g., by adhesives, stitches, staples, nails, clamps, and/or any other suitable fasteners). In the depicted example, platform 254 comprises a top plate 310 attached to a bottom plate 314, but in other examples, the platform may comprise a single plate, or more than two plates. In some examples, elastic inner wall 270 is attached to platform 254 by clamping lower end 286 of the elastic inner wall between top and bottom plates 310, 314. The elasticity of inner wall 270 allows platform 254 to be rotated and/or translated relative to base 210.

Platform 254 is configured to support an infant and to propagate low-frequency sound waves and/or vibrations (e.g., sound waves and vibrations having frequencies less than approximately 100 Hz). For example, platform 254 may be a thin, rigid expanse.

A mattress 320 may be disposed on platform 254 within interior 272. Mattress 320 is optional and may improve the comfort level of bassinet 200 for an infant lying in the bassinet.

A simulation module 350 is disposed on horizontal portion 220 of base 210. As described below, simulation module 350 includes components configured to perform motion-control and/or heartbeat-generation functions to simulate an intrauterine environment.

FIG. 6 is a side view depicting elastic inner wall 270 and platform 254 within base 210. Outer wall 264 is omitted from FIG. 5 for clarity. As FIG. 6 shows, simulation module 350 is disposed between horizontal base portion 220 and platform 254. In the example depicted in FIG. 6, simulation module 350 includes a box 356 having a box floor 364. Box 356 is open at the top, and box floor 364 is attached to horizontal base portion 220 by one or more bolts, screws, nails, staples, adhesives, and/or any other suitable fasteners. Simulation module 350 includes a motion-control assembly configured to simulate movement of the intrauterine environment (e.g., during a third trimester of gestation). An example motion-control assembly is described below.

A bass shaker 370 is attached to a bottom side 372 of platform 254. Bass shaker 370 is an example of transducer 174, described above. Accordingly, bass shaker 370 is configured to produce low-frequency sound waves and/or vibrations that propagate through platform 254 (e.g., from bottom side 372 toward an opposing top side 374, to an infant supported on the top side of the platform).

Optionally, a mid-range transducer 378 is also attached to bottom side 372. Transducer 378 is an example of mid-range transducer 188, described above.

C. Illustrative Simulation Assembly

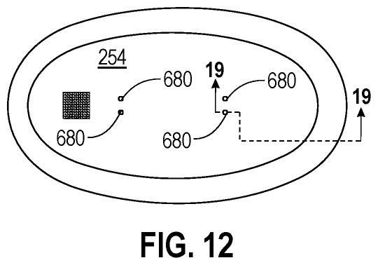

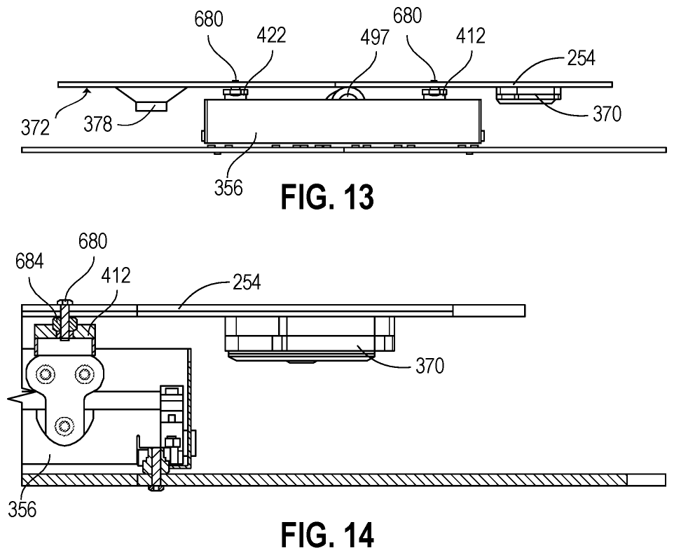

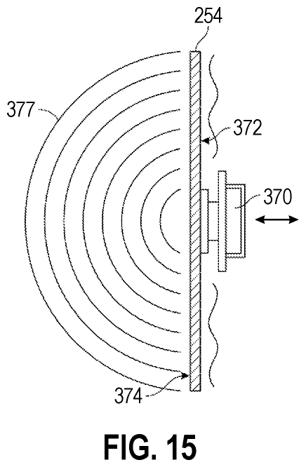

With reference to FIGS. 12-16, this section describes an illustrative simulation assembly 400 including a motion-control assembly 401. Simulation assembly 400 is an example of simulation module 350 suitable for use in bassinet 200, described above. Assembly 401 is an example of motion module 120, described above.

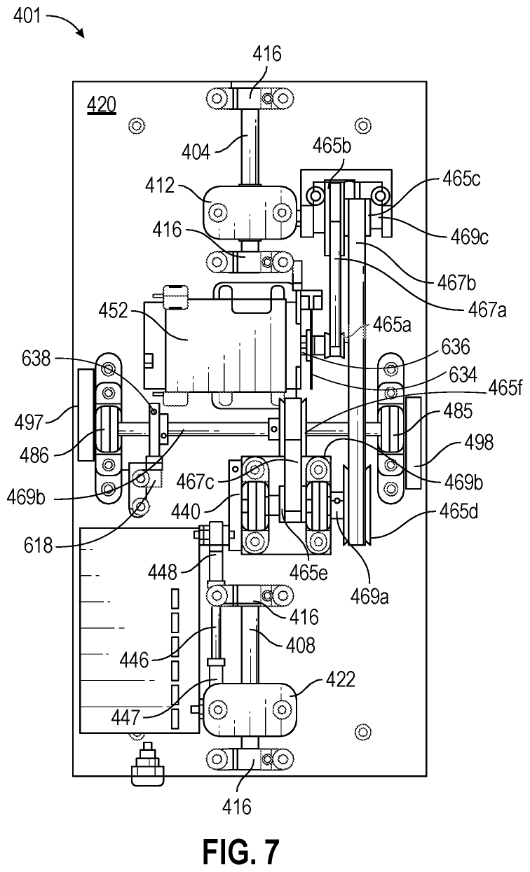

FIG. 7 is a plan view of motion-control assembly 401. Assembly 401 includes a robust suspension system comprised of first and second slide shafts 404 and 408 (also referred to as rods). First slide shaft 404 is slideably supported by a shaft support 412. A pair of rod fasteners 416 rigidly attach slide shaft 404 to a plate, indicated in FIG. 12 by the numeral 420. Depending on the embodiment, plate 420 may comprise a top or floor of a box of simulation module 350.

Second slide shaft 408 is slideably supported by a driving shaft support 422 and is rigidly attached to plate 420 by additional rod fasteners 416. Slide shafts 404, 408 are constructed from finely polished high-grade steel and/or any other material suitable for smooth motion and a long service life.

As described below, second slide shaft 408 is driven within driving shaft support 422, thereby linearly translating plate 420 (relative to another component, see below). First slide shaft 404 slides within support 412 as the plate is translated. In some examples, slide shafts 404, 408 are configured to be displaced relative to support 412 and driving support 422 respectively by approximately 1.5 inches (e.g., a displacement of approximately 0.75 inches in a first direction, and a displacement of approximately 0.75 inches in an opposing second direction). This corresponds to the average up-and-down displacement the infant experiences in the womb when the mother is walking. However, slide shafts 404, 408 may be configured for a different amount of displacement if desired. For example, a greater displacement may be used to better simulate the intrauterine environment of an infant whose mother is unusually tall. Additionally, or alternatively, the amount of displacement may be varied over time. For example, the displacement may be decreased over the weeks or months following the infant's birth, thereby acclimating the infant to the extrauterine environment.

Typically, in the womb, the infant is oriented in an approximately vertical position (e.g., with its head substantially toward or away from the ground when the mother is standing), but after being born the infant typically sleeps and rests in a horizontal position. Accordingly, assembly 401 is typically oriented (e.g., within bassinet 200 or other suitable structure) such that slide shafts 404, 408 move in a horizontal plane (e.g., a plane substantially coplanar with the ground during normal use).

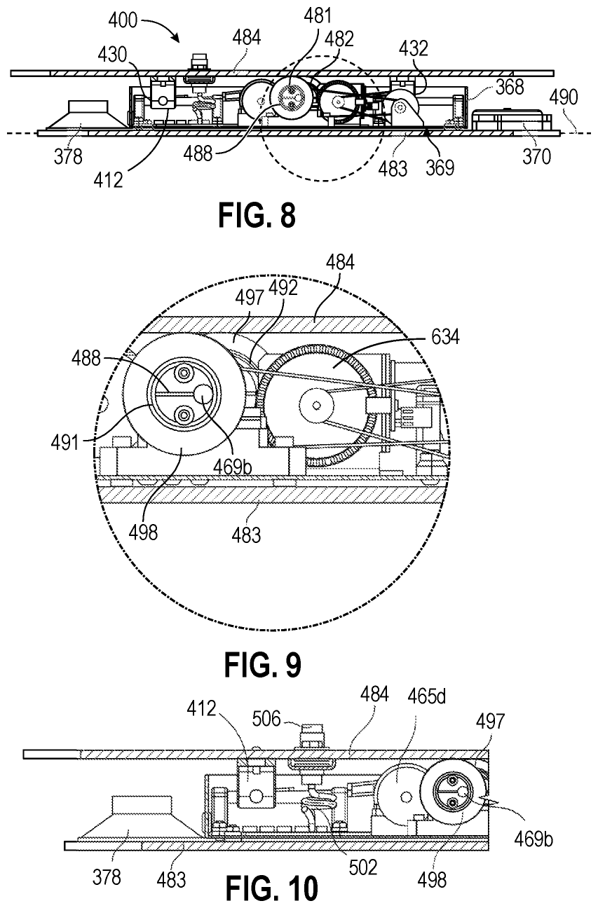

First slide shaft 404 is slideably supported within shaft support 412 by at least one bushing 430, and second slide shaft 408 is slideably supported within driving shaft support 422 by at least one bushing 432 (see FIG. 8). Bushings 430, 432 enable shafts 404, 408 to rotate within respective supports 412, 422. Rotational motion of assembly 401 is described below.

Driving shaft support 422 is operatively coupled to a cam pulley 440 via a driving rod 446 having rod end linkages 447, 448. A motor 452 is configured to drive cam pulley 440 via a plurality of pulleys 465A-465E, belts 467A-467C, and shafts 469A-469C. These pulleys and belts can be configured in various ratios to provide different levels of torque and RPM depending on the motor type and size. The number and arrangement of pulleys, belts, and shafts in FIG. 7 is an illustrative example, and any suitable assembly for driving second slide shaft 408 and/or first slide shaft 404 may be used.

Assembly 401 further includes a pair of offset cams 481, 482 (see FIG. 8) configured to cause rotational motion. Depending on the installation of simulation module 350, offset cams 481, 482 may either impart rotational motion to plate 420, or may rotate another platform relative to plate 420. FIGS. 8-10 depict an example wherein assembly 401 is disposed substantially within open-bottomed box 368. Box top 369 is an example of plate 420 indicated in FIG. 12. Box top 369 is disposed adjacent and/or engaging a top plate 483. Offset cams 481, 482 protrude from the open bottom of the box to push a bottom plate 484. Shaft support 412 and driving shaft support 422 are rigidly attached to bottom plate 484. Typically, the infant would be supported above top plate 483.

Offset cams 481, 482 are attached to shaft 469B. Shaft 469B rotates on a pair of bearing pillow blocks 485, 486 and is driven by pulley 465F and belt 467C, which is powered from pulley 465E. Bearing pillow blocks 485, 486 are rigidly attached to box top 369. Additionally, or alternatively, the bearing pillow blocks may be rigidly attached to top plate 483.

Offset cams 481, 482 each have a lobe 488 offset from a center of shaft 469B by a predetermined distance. The predetermined distance may be within a range of 0 to 15 millimeters (mm), 5 to 10 mm, and/or any other suitable range. In the depicted example, the distance is approximately 7.8 mm, which corresponds to an angular displacement of +/−4.5 degrees rotation. Offset cams 481, 482 are mounted on shaft 469B, which is oriented substantially transverse to a longitudinal axis 490 defined by plate 483. Offset cams 481, 482 are offset from each other by 180 degrees, such that when one of the cams is at its highest position, the other cam is at its lowest position. Accordingly, rotating the cams together (e.g., by rotating shaft 469B) causes plate 483 to rotate about longitudinal axis 490 defined by the plate.

Each of offset cams 481, 482 has a respective ball bearing 491, 492 pressed over its outer surface. A respective tire 497, 498 made from a resilient plastic or rubber material, and/or the like, is press-fitted over each ball bearing 491, 492. Mechanical linkages of assembly 401 are designed such that both tires 497, 498 have a slight pressure on the bottom plate 484 and are in continuous contact with it. The arrangement of two offset cams, one on each side of longitudinal axis 490, allows a very smooth rocking action.

The ratio between the longitudinal motion cycle and the rotational motion cycle is 2:1. In other words, for each full cycle of cam pulley 440, the platform only rotates a half-cycle. This 2:1 ratio is based on the motion of the mother's hips (see FIG. 1). Typically, assembly 401 does not allow the ratio to be varied, but in some examples, the ratio may be variable. The ratio is determined by pulleys 465E and 465F.

Alternatively, as described above, assembly 401 may be disposed within open-top box 356. FIG. 6, described above, depicts such an example. In the example depicted in FIG. 6, floor 364 of box 356 is rigidly attached to horizontal base portion 220, and offset cams 481, 482 (e.g., tires 497, 498 attached to the cams) extend from the open top of the box to push against platform 254. The offset of the cams causes platform 254 to rotate relative to horizontal base portion 220. Pillow bearing blocks 485, 486 are rigidly attached to box floor 364 and/or to horizontal base portion 220. The sliding shafts and associated supports may be configured in any manner suitable to enable motion of platform 254 relative to base 210. For example, shaft supports 412, 422 may be rigidly attached to box floor 364 and/or to horizontal base portion 220, with slide shafts 404 and 408 rigidly attached to bottom side 372 of platform 254.

FIG. 10 depicts an interior power cable 502 within box 368 and configured to supply power to motor 452 and/or any other suitable components of assembly 400 (e.g., an electronic controller, bass shaker 370, mid-range transducer 378, one or more sensors, etc.). Interior power cable 502 is configured to be coupled to an external power cable by a power connector 506.

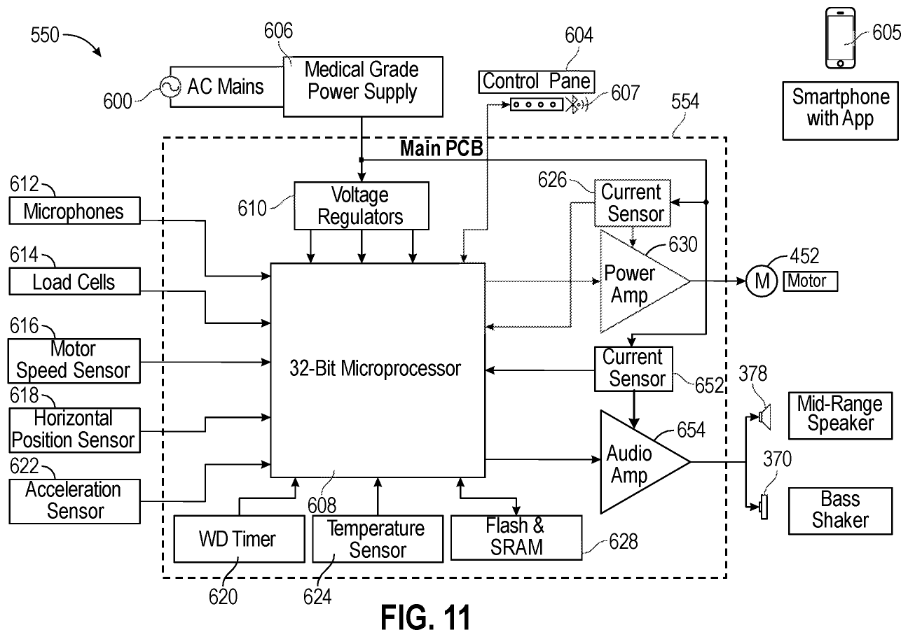

FIG. 11 schematically depicts an illustrative control system 550 in accordance with aspects of the present teachings. Control system 550 is an example of a control system 150, described above.

Electrical power is delivered to bassinet 200 (or another suitable device) through control system 550. An external power source, such as an AC mains power 600, is coupled to a medical-grade low-voltage DC power supply 606 (e.g., through power cable 502 and connector 506, described above with reference to FIG. 10).

A control panel 604 disposed on or adjacent power supply 606 provides a user interface configured to allow a user to turn the power supply to the bassinet on or off, to start or stop motor 452, and/or to change other settings of the bassinet (based on a time of day or night, an age of the infant, etc.). In some examples, control panel 604 is a backup control panel intended to be used primarily when a main controller (such as a smartphone 605, remote control, computer, or other suitable device) is unavailable.

Control panel 604 may be disposed on any suitable location of bassinet 200 or another suitable device. For example, control panel 604 may be disposed on base 210 of bassinet 200.

Control system 550 is configured to receive and/or transmit communications (e.g., to smartphone 605) via a Bluetooth wireless communication system 607. In some examples, communications may additionally or alternatively be made via a WiFi wireless communications system, any other adequate wireless communications means, or a dedicated wired communications connection.

In some examples, power supply 606 and wireless communication system 607 are located at a distance from interior 272 of bassinet 200, thereby reducing potential and/or perceived hazards from high voltages, electromagnetic fields, and/or high frequency radiated energy.

Control system 550 includes a main printed circuit board assembly 554 (also called a PCB or PCBA) including a processor 608. In the depicted example, processor 608 comprises a 32-bit microprocessor, but in other examples, any suitable processor or processors may be used. One or more voltage regulators 610 regulate the voltage supplied to processor 608 by power supply 606.

A plurality of sensors are coupled to and/or in communication with processor 608. In the depicted example, sensors coupled to processor 608 include a microphone 612, load cell(s) 614, motor speed sensor 616, horizontal position sensor 618, watchdog timer 620, acceleration sensor 622, temperature sensor 624, and current sensors 626 and 652.

Current sensor 626 in combination with acceleration sensor 622 is monitored and logged to provide real-time safety. If motor 452 malfunctions and produces excess current, or if the entire platform experiences an unexpected or sudden movement (e.g., acceleration), sensors 622, 626 signal processor 608 to quickly halt the motor drive. For example, a dangerous situation could occur if the infant's toddler sibling climbed onto bassinet 200 while motor 452 is operating, and sensors 622, 626 could detect this situation and signal processor 608 to stop the motor. In some examples, sensors 622, 626 are configured to log any abnormal activity, and/or to periodically log normal activity (e.g., in a flash and/or SRAM memory store 628). Logged data relating to normal and/or abnormal activity can be used to predict equipment failures and help guide preventative maintenance.

Microphone(s) 612 is configured to monitor sounds in and/or adjacent bassinet 200 (e.g., sounds made by an infant within the bassinet). Processor 608 is configured to analyze sounds received by microphone 612 (e.g., in real-time) to detect occurrences of crying, cooing, and/or the like. In response to detecting crying (and/or another type of sound), a record may be made in memory store 628. In some examples, the record comprises an indication that crying occurred, and may further comprise data associated with the time at which the crying was recorded.

Additionally, or alternatively, an audio recording of the actual detected sound may be stored. In some examples, microphone 612 is omitted.

Load cells 614 enable weighing of the infant. Processor 608 is configured to monitor outputs from the load cells and to compare output corresponding to an empty platform to output corresponding to the presence of an infant to determine the infant's weight. The weight is logged (e.g., in memory 628). Memory 628 may be accessible via smartphone 605 and/or any other suitable device in communication with the memory.

Watchdog timer 620 is configured to monitor the execution of instructions (e.g., code) by processor 608. Code correctly run by processor 608 will reset watchdog timer 620 on a regular interval. Failure of watchdog timer 620 to reset within a predetermined time period typically indicates a system error. If watchdog timer 620 is not reset in the expected amount of time, then the timer times out and causes a reboot of processor 608 and/or other components of system 550.

Temperature sensor 624 is included on main PCB 554 as a safety feature configured to detect over-temperature conditions, which are expected never to occur during normal operation. In response to detection of an excessive temperature by temperature sensor 624, processor 608 may be configured to activate an alarm, halt operation of motor 452, and/or perform any other suitable action.

Control system 550 is configured to control motor 452. A power amplifier 630 is coupled to processor 608 and to motor 452 and configured to amplify a signal from the processor to control the speed (e.g., RPM) of the motor. Processor 608 determines a suitable target speed for the motor based on a current mode of operation of assembly 401 (e.g., ramping up, ramping down, steady speed, night mode, day mode, etc.). The mode of operation may be settable by a user via control panel 604 and/or smartphone 605. Based on the mode of operation, processor 608 drives motor 452 at an appropriate level.

Processor 608 is coupled to an encoder disk 634 attached to a shaft 636 of motor 452 (see FIG. 12). Processor 608 is configured to receive data from encoder disk 634 representing a speed of rotation of shaft 636. Motor speed sensor 616 of control system 550 is configured to measure a time interval between pulses of encoder disk 634. Based on the data received from disk 634 and the time measured by motor speed sensor 616, processor 608 determines a speed of motor 452 and, as needed, adjusts a supply of power to the motor such that a target speed of the motor is maintained. This enables motor 452 to be operated at the target speed irrespective of changes in position of the infant, changes in weight in the bassinet, and/or any other factors.

When movement of platform 254 is stopped, it is desirable that the platform come to rest in a horizontal position (e.g., substantially level with the ground), so that the infant supported by the platform does not roll. Processor 608 is configured to stop motor 452 in response to receiving information from horizontal position sensor 618 indicating that the platform is level. Horizontal position sensor 618 may comprise any suitable sensor configured to detect whether the platform is level. In the depicted example, horizontal position sensor 618 is configured to sense a presence and/or position of a horizontal position indicator 638 (see FIG. 12) which is mounted on a circumference of cam pulley 440. Indicator 638 may comprise any device detectable by horizontal position sensor 618. For example, sensor 618 may comprise a Hall-effect sensor, and indicator 638 may comprise a magnet. Additionally, or alternatively, sensor 618 may be configured to detect indicator 638 using a simple mechanical switch, optically and/or via RFID. Yet another alternative or additional approach is to detect a horizontal position using software, following a calibration step.

Processor 608 is further configured to control mid-range transducer 378 and bass shaker 370 (if included). Typically, processor 608 is configured to drive an audio amplifier 654, which is configured to drive mid-range transducer 378 and optional bass shaker 370. Amplifier 654 amplifies audio waveforms supplied by processor 608. In some examples, respective dedicated amplifiers are configured to drive mid-range transducer 378 and bass shaker 370.

A current sensor 652 is configured to monitor current levels within audio amplifier 654. Current sensor 652 is coupled to processor 608. In response to a detection of excessive current levels within amplifier 654 by current sensor 652, processor 608 may be configured to sound an alarm, stop a power supply to the amplifier, and/or take any other suitable action. In this manner, current sensor 652 prevents mid-range transducer 378 and bass shaker 370 from exceeding a predetermined cutoff power level.

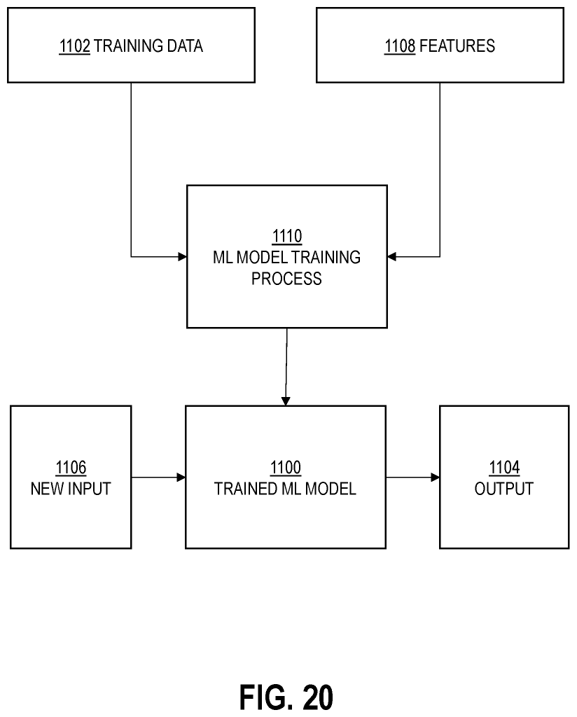

As described above, processor 608 and amplifier 654 drive bass shaker 370 at relatively low frequencies (e.g., predominantly in a range of 20 to 80 Hz), such that low-frequency vibrations propagate from bass shaker 370 through the attached platform (e.g., plate 483 and/or platform 254) in the manner depicted in FIG. 20 (e.g., in planar dispersion pattern 377). This provides an infant on the platform with a simulated substantially full-body intrauterine heartbeat experience.

To enable platform 254 to radiate low-frequency waves accurately (e.g., without significant distortion and/or attenuation), the platform is thin, firm, and configured to be isolated from non-moving masses likely to distort and/or attenuate the waves. Accordingly, one or more vibration-isolating components may be used to connect the platform to which bass shaker 370 is attached to motion-control assembly 401.

FIGS. 12-14 depict bass shaker 370 and mid-range transducer 378 attached to underside 372 of platform 254. A plurality of fasteners 680 attach shaft support 412 and driving shaft support 422 to platform 254. Each fastener 680 passes through a respective isolation grommet 684. Isolation grommet 684 vibrationally isolates platform 254 (to which the transducers are attached) from box 356 (within which motion-control assembly 401 is substantially disposed). Isolation grommets 684 may comprise any suitable material for reducing and/or substantially preventing vibrational coupling between box 356 and platform 254. For example, isolation grommets 684 may comprise a low-durometer elastomeric material.

In other examples, isolation grommets 684 may be omitted and vibration isolators comprising another form and/or material may be used. Suitable vibrationally isolating devices may include pieces of rubber, cork, foam, and/or laminate; mechanical springs or spring dampeners; tuned mass dampeners; pneumatic isolators; and/or the like.

D. Illustrative Cushion

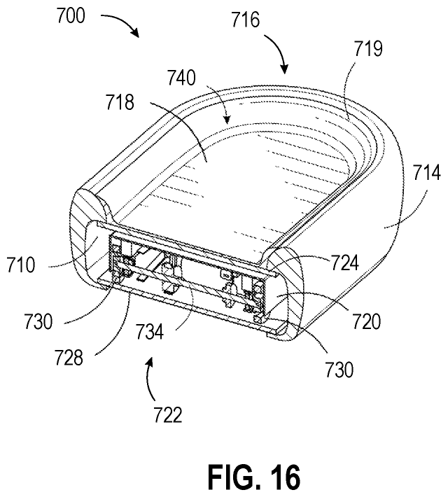

With reference to FIG. 16, this section describes an illustrative cushion 700 configured to simulate an intrauterine environment for an infant placed on the cushion. Cushion 700 is another example of a system configured to simulate an intrauterine environment, described above.

FIG. 16 is an isometric cutaway view depicting cushion 700. Cushion 700 has an interior cavity 710 at least partially encased in a resilient exterior 714. Resilient exterior 714 may comprise foam, down, microbeads, hollow silicone rubber, and/or any other material suitable for comfortably supporting an infant and allowing for longitudinal and rotation motions produced by a motion-control assembly.

An upper portion 716 of exterior 714 may include a depressed central portion 718 disposed within a raised perimeter portion 719. An infant may be placed in depressed central portion 718 and raised portions 719 may help to prevent the infant from rolling off of cushion 700, or from dropping items off the side of the cushion. Additionally, or alternatively, central portion 718 has relatively little (relative to raised portions 719) or no cushioning underneath, and therefore may propagate low-frequency acoustic waves and/or vibrations produced by a bass transducer with relatively low attenuation. However, an infant may be placed on cushion 700 in any suitable way.

A simulation module 720 is disposed within cavity 710. In the depicted example, module 720 includes a motion-control assembly 722 disposed between a top platform 724 and a bottom platform 728, and may further include a bass shaker (not shown) and/or a mid-range transducer (not shown). Motion-control assembly 722 may be substantially similar to motion-control assembly 401, described above. For example, as shown in FIG. 16, assembly 722 includes a pair of offset cams 730 mounted to a shaft 734.

Exterior 714 encloses module 720 for safety and aesthetic reasons. Exterior 714 may include a substantially waterproof liner 740 configured to prevent ingress of fluids into cavity 710. Liner 740 is optional and may be omitted. In some examples, exterior 714 comprises a waterproof material (e.g., silicone rubber) even without a liner.

E. Illustrative Infant Data Collection System

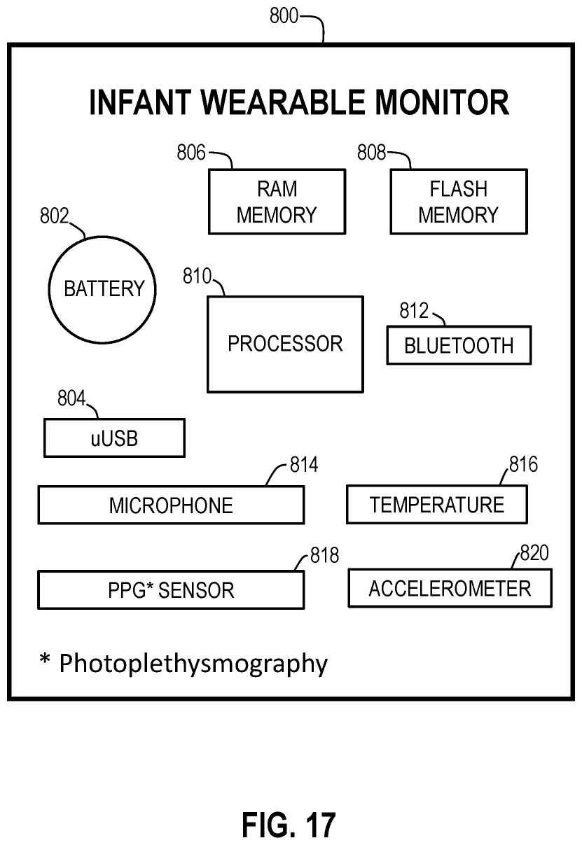

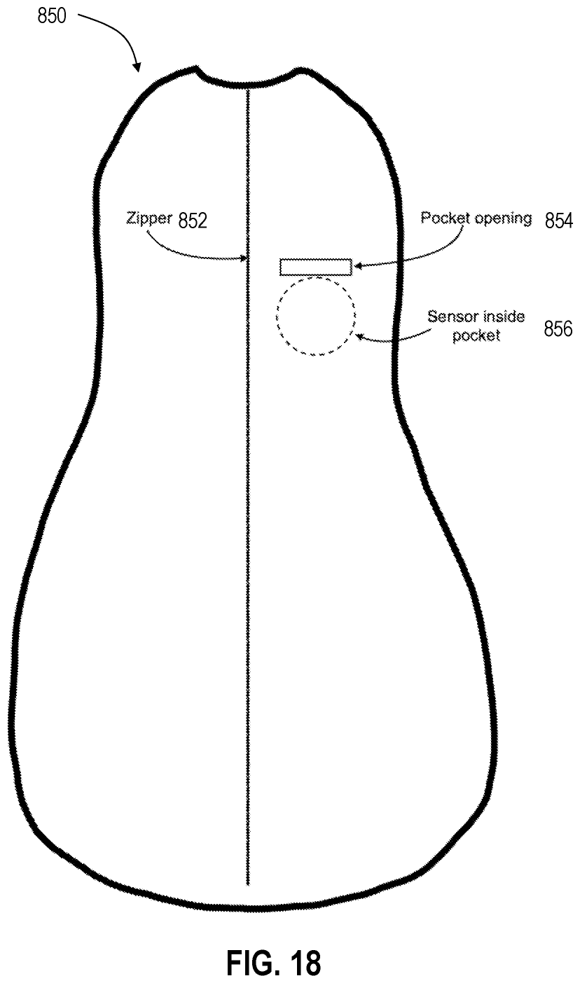

With reference to FIGS. 17 and 18, this section describes an illustrative system 800 for the collection of infant data. System 800 includes a wearable monitor having a plurality of sensors, such as a microphone 814, a temperature sensor 816, a photoplethysmography sensor 818, and/or an accelerometer 820. System 800 includes a power supply in the form of a battery 802 configured to power the various sensors, and a Bluetooth or other wireless communication device 812 for wirelessly communicating with other systems and/or monitoring devices (e.g., a mobile phone). Data collected by system 800 may be sent to an electronic controller of the intrauterine simulation system to be used in determining operating parameters and/or providing feedback regarding effects of the settings already in use. As depicted in FIG. 18, system 800 may be coupled to or otherwise incorporated into an article of clothing 850. In the depicted example, system 800 may be incorporated or coupled to the clothing at various locations including a zipper area 852, an outside pocket 854, and an inside pocket 856.

F. Illustrative Data Analysis of Biometric Data and Use in Intrauterine Simulation Devices

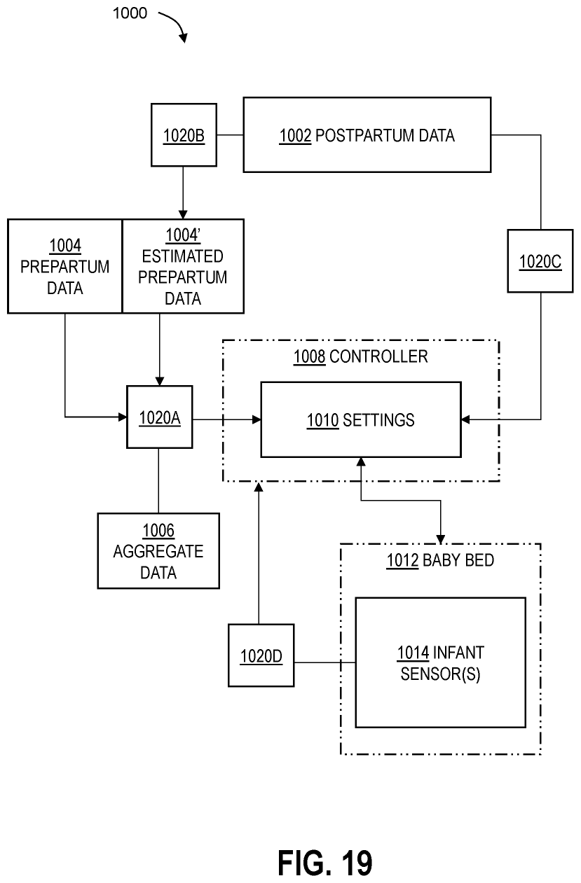

Turning to FIGS. 19 and 20, systems and algorithms for determining and modifying settings of intrauterine simulation systems such as those described herein (e.g., system 100, 200, 400). FIG. 19 is a schematic diagram depicting a custom biometric data transformation system 1000, suitable for use with systems of the present disclosure.

System 1000 includes an intrauterine simulation device 1012 in communication with an electronic controller 1008, where the controller is configured to apply one or more settings 1010 (AKA parameters) to device 1012 to control and modify the intrauterine simulation. Settings 1010 may include numerical values and/or categories. For example, several predetermined walking gait categories may be available for simulation by a mechanical actuator system of device 1012, and one of those categories may be selected automatically by controller 1008. In some examples, controller 1008 determines (or receives) and applies a heartrate setting to device 1012 to control the rate at which the heartbeat is simulated. In some examples, settings 1010 may include other selectable categories, such as Fast/Slow, Gentle/Average/Robust, etc.

An infant may be supported in device 1012, and one or more infant sensors 1014 may be associated with the infant. For example, the infant may have a wearable sensor, such as system 800 described above. Feedback from infant sensors 1014 (e.g., infant heartrate, noise, motion, etc.) may be utilized to automatically adjust settings 1010. For example, an increase in infant heartrate may indicate distress, leading to an automatic reduction (or increase) in volume for an audio speaker and/or a change (increase or decrease) in the motion of device 1012. Other sensors may be present on device 1012, as described above, and may be utilized to monitor and adjust device operation.

In some examples, prepartum data 1004 collected from the mother of the infant is utilized to determine appropriate or desired settings 1010. Prepartum data 1004 may be collected using any suitable methods and/or devices, such as by having the mother wear a sensor-enabled wristwatch or other wearable device (e.g., an Apple watch or a FitBit device) and/or self-reporting, guided interview (e.g., via an app or web interface). Prepartum data 1004 may include data on any suitable variable, such as heartrate, activity level vs. time, step count, gait length, sleep quality, sleep duration, bedtime, waking time, exercise frequency, duration, and/or intensity, sound recordings, and/or the like. Whether data is collected automatically (e.g., using a device), or collected manually (e.g., via surveys), the gathered information may be preprocessed into a format suitable for storage in one or more databases or data stores (see below).

Using a conversion module 1020A, some or all of the mother's prepartum data 1004 may be converted into settings 1010 appropriate for a child of the mother. Conversion module 1020A may include any suitable processing logic and/or software configured to receive some or all of prepartum data 1004 as inputs and to determine, based on the inputs, one or more values to be output to (and utilized by) controller 1008 as settings 1010. In some examples, the output of conversion module 1020A is utilized directly by one or more aspects of device 1012 (e.g., when controller 1008 is not present, or is implemented in a distributed manner). In some examples, conversion module 1020A is a machine learning model (see below) trained to determine one or more settings 1010 based on prepartum data 1004. Conversion by module 1020A from prepartum data 1004 to machine settings 1010 may include correlations generated by a machine learning algorithm and/or by an explicitly programmed algorithm such as a lookup table or if-then analysis. Conversion module 1020A may incorporate or be in communication with or be trained based on a combined dataset including prepartum biometric data for a cohort of females (i.e., mothers) and corresponding settings.

In some examples, e.g., when the mother's prepartum data is unavailable, aggregated prepartum data from a cohort of female subjects is utilized to determine appropriate or desired settings 1010. With the goal of customizing cradle operation for a specific mother-baby pair, aggregated data may still be used. A model mother may be constructed having similar characteristics to the actual mother whose prepartum data is unavailable. Data from the cohort of mothers can be divided into subcategories that may include age, weight, health, activity level, race, etc. The data characteristics may thereby be tuned to those of the infant's own mother. By using this data set and selection algorithm, mothers with unknown prepartum data may nonetheless have a unique set of parameters developed for their babies. The aggregated data may be input to conversion module 1020A, which is configured to determine one or more of settings 1010 as described above.

In some examples, e.g., when the mother's prepartum data is unavailable, postpartum data 1002 is collected from the mother. Postpartum data 1002 may be collected using any suitable methods and/or devices, similar to prepartum data 1004. In some examples, both prepartum data 1004 and postpartum data 1002 are collected from the same mother of the baby. A collection of such prepartum and postpartum data from a cohort of mothers can be used to inform explicit conversion algorithms or to train machine learning algorithms (see below), such as conversion module 10208. Conversion module 10208 may include any suitable processing logic and/or software configured to receive some or all of postpartum data 1002 as inputs and to determine, based on the inputs, estimated prepartum data 1004′. Estimated prepartum data 1004′ can then be input to conversion module 1020A to generate one or more settings 1010. Conversion by module 1020B from postpartum data 1002 to estimated prepartum data 1004′ may include correlations generated by a machine learning algorithm and/or by an explicitly programmed algorithm such as a lookup table or if-then analysis. Conversion module 10208 may incorporate or be in communication with or be trained based on a combined dataset including both prepartum and postpartum biometric data for a cohort of females (i.e., mothers).

In some examples, one or more settings 1010 are determined directly from postpartum data 1002, using a conversion module 1020C. Conversion module 1020C may include any suitable processing logic and/or software configured to receive some or all of postpartum data 1002 as inputs and to determine, based on the inputs, one or more settings 1010. For example, conversion module 1020C may include a machine learning algorithm trained to receive postpartum data 1002 as inputs and to output a walking gait category (and/or other setting 1010) that would correspond to the mother's prepartum data if it were available. Conversion by module 1020C from postpartum data 1002 to machine settings 1010 may include correlations generated by a machine learning algorithm and/or by an explicitly programmed algorithm such as a lookup table or if-then analysis. Conversion module 1020C may incorporate or be in communication with or be trained based on a combined dataset including postpartum biometric data of the mother of the baby and/or for a cohort of females (i.e., mothers) and corresponding settings.

In some examples, data from infant sensor(s) 1014 is utilized to provide feedback to controller 1008, such that settings 1010 can be dynamically adjusted in response to infant behavior. For example, information from the infant sensors may be received as input by a conversion module 1020D, which in turn outputs one or more signals to the controller to adjust settings 1010, e.g., by reducing or increasing a magnitude of one of the simulation effects. In some examples, data from the infant sensors is communicated directly to the controller. In some examples, output from conversion module 1020D directly controls settings 1010. For example, conversion module 1020D may respond to certain inputs by implementing a global reduction or increase in some or all settings 1010. Conversion module 1020D may include any processing logic and/or software configured to respond to infant sensor data by adjusting one or more settings 1010 and/or outputting commands or recommendations to controller 1008 regarding settings 1010.

Accordingly, data may be utilized in system 1000 via one or more of several pathways. In a first pathway, prepartum data 1004 from the mother of the baby is converted to one or more settings 1010 using conversion module 1020A. In a second pathway, aggregated data 1006 from a cohort of mothers (including or not including the actual mother) is converted to one or more settings 1010 using conversion module 1020A. In a third pathway, postpartum data 1002 from the mother of the baby is converted to estimated prepartum information 1004′ using conversion module 10208, and then to one or more settings 1010 using conversion module 1020A. In a fourth pathway, postpartum data 1002 from the mother of the baby is converted directly to one or more settings 1010 using conversion module 1020C. In a fifth pathway, infant data 1014 (e.g., real time infant data) is converted to commands provided to controller 1008 for affecting one or more settings 1010 and/or to commands directly affecting the setting(s), using conversion module 1020D.

Although conversion modules 1020A-D are referred to in similar terms, each of the conversion modules (a) may or may not be present or utilized, and (b) may be implemented in a different manner using different algorithms. For example, conversion module 1020C may include a trained neural network, while conversion module 1020D may include an explicitly programmed decision tree. Furthermore, any or all of the data discussed above may be located on a computer network (e.g., in the cloud). Any or all of the conversion modules may be disposed on-device (e.g., as part of controller 1008) or on a network server (e.g., in the cloud). In some examples, conversion module 1020D is included either in the infant sensor system or in controller 1008. Communication between modules and the controller may be carried out wirelessly and/or over a network (e.g., over the Internet).

FIG. 20 depicts the training and use of an illustrative machine learning algorithm or model 1100. As mentioned above, machine learning algorithms may be utilized in one or more conversion aspects of system 1000 (and/or other systems described herein).

In general, machine learning (ML) models (AKA ML algorithms, ML tools, or ML programs) may be utilized to generate predictions or decisions that are useful in themselves and/or in the service of a more comprehensive program. ML algorithms “learn” by example, based on existing sample data, and generate a trained model. Using the trained model, predictions or decisions can then be made regarding new data without explicit programming. Machine learning therefore involves algorithms or tools that learn from existing data and make predictions about novel data.

Training data 1102 (e.g., labeled training data) is utilized to build trained ML model 1100, such that the ML model can produce a desired output 1104 when presented with new data 1106. In general, the ML model uses labeled training data 1102, which includes values for the input variables and values for the known correct outputs, to ascertain relationships and correlations between variables or features 1108 to produce an algorithm mapping the input values to the outputs.

Supervised learning methods may be utilized for the purposes of producing classification or regression algorithms. Classification algorithms are typically used in situations where the goal is categorization (e.g., whether a photo contains a cat or a dog). Regression algorithms are typically used in situations where the goal is a numerical value (e.g., the market value of a house).

Features 1108 may include any suitable characteristics capable of being measured and configured to provide some level of information regarding the input scenario, situation, or phenomenon. For example, if the goal is to provide an output relating to the market value of a house, then the features may include variables such as square footage, postal code, year built, lot size, number of bedrooms, etc. Although these example features are numeric, other feature types may be included, such as strings, Boolean values, etc.

Different ML techniques may be used, depending on the application. For example, artificial neural networks, decision trees, support-vector machines, regression analysis, Bayesian networks, genetic algorithms, random forests, and/or the like may be utilized to produce the trained ML model.

Trained ML model 1100 is produced by training process 1110 based on identified features 1108 and training data 1102. Trained ML model 1100 can then be utilized to predict a category or decide an output value 1104 based on new data 1106.

With respect to the present disclosure, ML models may be used at various points in the data processing algorithm(s). For example, a first ML model may be utilized to determine prepartum data based on postpartum data, e.g., in conversion module 1020B. In this example, the training data includes prepartum and postpartum biometrics of a cohort of mothers, such that the ML model can predict prepartum results based on postpartum inputs. In another example, a second ML model may be utilized to determine one or more settings of the intrauterine simulation, such as gait type or active times of the day, based on postpartum data, e.g., in conversion module 1020C. In this example, the training data includes postpartum inputs and machine setting outputs. This training data may be a combination of the postpartum/prepartum data described above and corresponding device settings known to be appropriate.

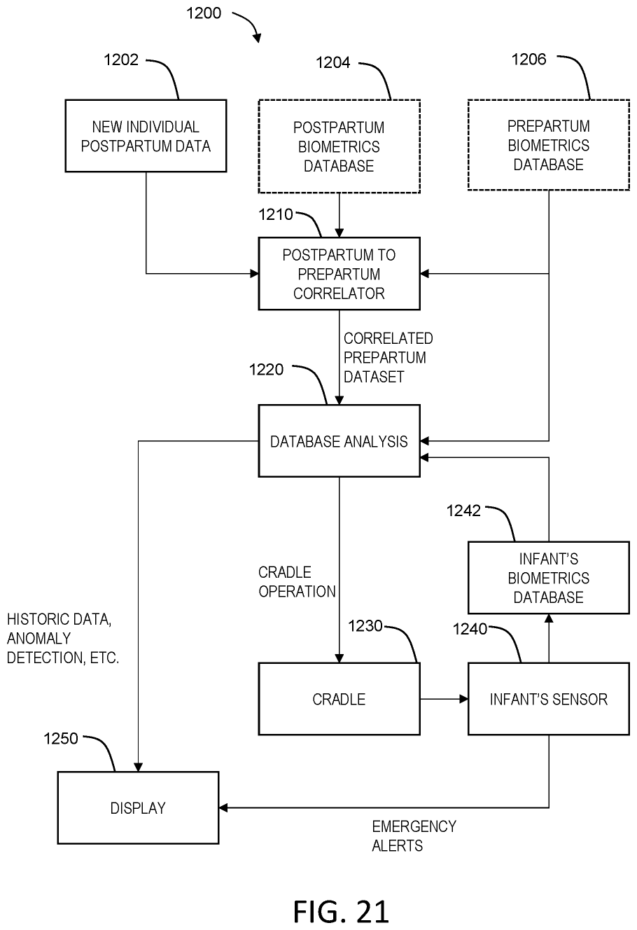

FIG. 21 depicts a schematic diagram of a system 1200, which is an example of system 1000 described above. As depicted in this example, some or all of several datasets may be included: the mother's prepartum biometrics database 1206, the mother's postpartum biometrics database 1204, and a new individual postpartum data set 1202. Databases 1204 and 1206 including a cohort of mothers are used to generate a postpartum to prepartum correlator that may be utilized to correlate the new individual postpartum data to an estimated prepartum set 1210. The estimated prepartum set or the mother's prepartum biometrics database 1206 may be analyzed and converted to parameters usable by the intrauterine simulation system 1220. The parameters may be sent to an intrauterine simulation system to determine operation settings 1230. In this example, operation settings 1230 correspond to settings 1010 in system 1000.

An infant sensor 1240 may be used to record data for an infant biometric database 1242 configured to provide feedback that may be analyzed and sent to the intrauterine simulation system 1230 to adjust operation settings. Both historic data, anomaly detection, and the recordings for the infant sensor 1240 may be sent to a display 1250 (e.g., of a handheld device or desktop computer).

G. Illustrative Methods for Biometric Data Collection

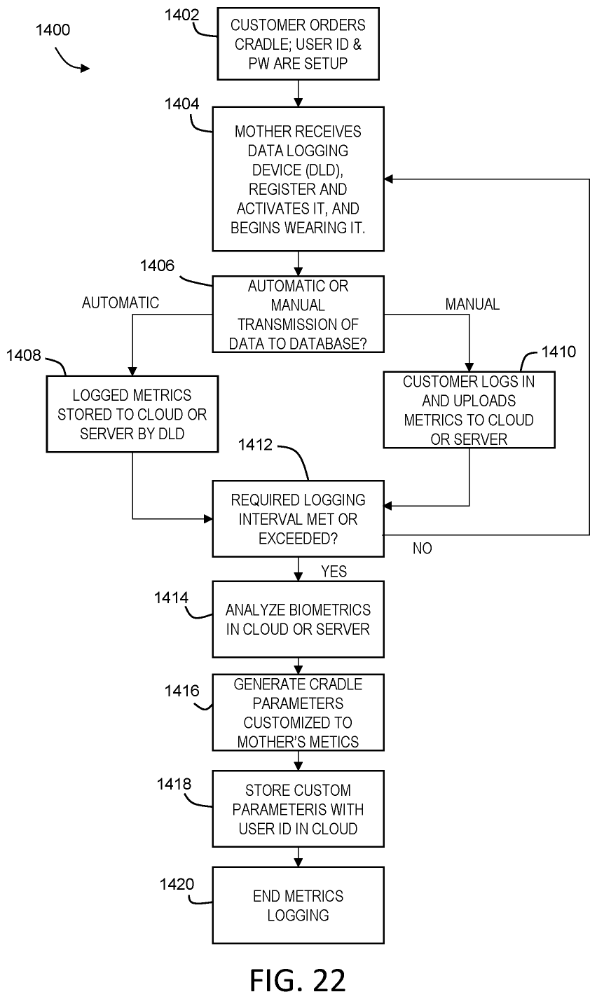

With reference to FIGS. 22-24, this section describes steps of an illustrative method 1400 for collecting biometric data to be used by the intrauterine simulation system and using the system. Aspects of the intrauterine simulation system already described may be utilized in the method steps described below. Where appropriate, reference may be made to components and systems that may be used in carrying out each step. These references are for illustration and are not intended to limit the possible ways of carrying out any particular step of the method. Method 1400 is intended to provide illustrative, supplemental, and/or alternative steps with respect to those described above with respect to system 1000 and system 1200.

FIGS. 22-24 are flowcharts illustrating steps performed in method 1400 and may not recite the complete process or all steps of the method. Although various steps of method 1400 are described below and depicted in FIGS. 22-24, the steps need not necessarily all be performed, and in some cases may be performed simultaneously or in a different order than the order shown.

As shown in FIG. 22, method 1400 starts when a customer, who may be a mother during prepartum or another who is associated with a prepartum mother, orders a cradle and sets up a user identification (ID) and password (PW) 1402. The mother may then receive a data logging device such as a fitness tracker, smart watch, or similar device, to record her biometrics at block 1404. Recorded data may include aspects of biometric data including but not limited to heartrate, respiration rate, oxygen level, activity level, sleep states, etc. Recorded data may also include age, weight, height, ethnicity, education level, geographical location, diet, infant's gestation period, APGAR scores, and/or the like.

The data logging device may be set to automatically or manually transmit recorded data to the database in the cloud or server at block 1406. If set to manually transmit data, the customer logs in to the secure website and uploads the biometrics at block 1410. If set to automatically transmitted, the logged data is stored to the cloud or server 1408 by DLD. The transmission setting may be changed at any point within the required logging interval. Once the required logging interval has be met or exceeded at block 1412 the biometrics may be analyzed in the cloud or server at block 1414 and used to generate intrauterine simulation system parameters at block 1416. The custom parameters may be stored in the cloud with the customer's user ID at block 1418, ending the metrics logging at block 1420.

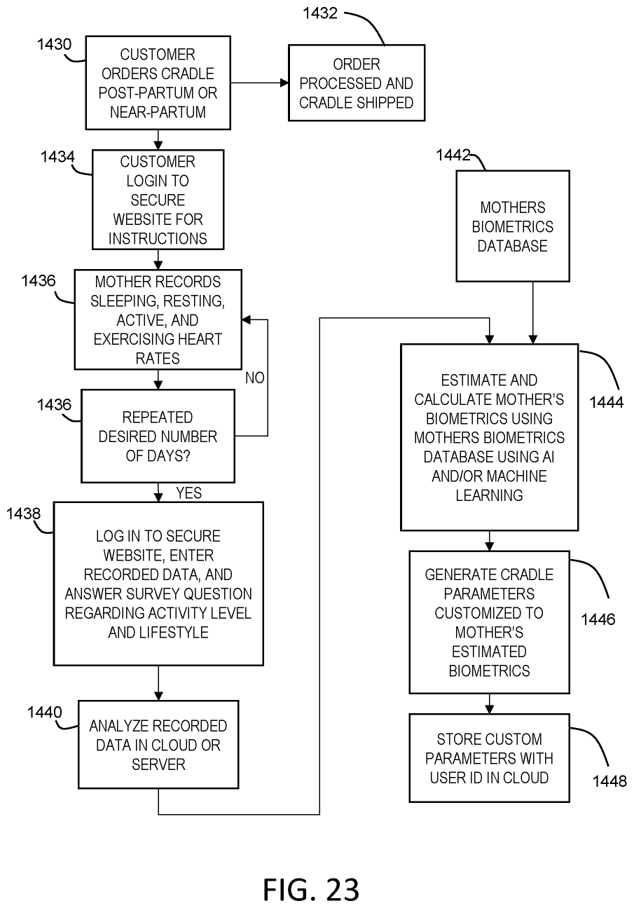

An alternative scenario, where the mother is postpartum, is shown in FIG. 23. Upon ordering an intrauterine simulation system at block 1430, a customer may log on to a secure website for instructions on recording biometric data at block 1434. Recorded data may include aspects of biometric data similar to that of the prepartum scenario or may include other aspects. Recording of data lasts for a desired number of days at block 1436. Once the desired number of days has been reached at block 1436, the data may be entered in the secure website and a short survey featuring questions such as activity level and lifestyle habits may be filled out at block 1438.

Recorded data entered into the website may be analyzed in the cloud or in a server at block 1440 and may be converted by an algorithm at block 1444. The data may then be converted into parameters usable by the intrauterine simulation system that simulates the biometrics of the mother at block 1445. The data may be stored in the cloud or server for current or later use at block 1448.

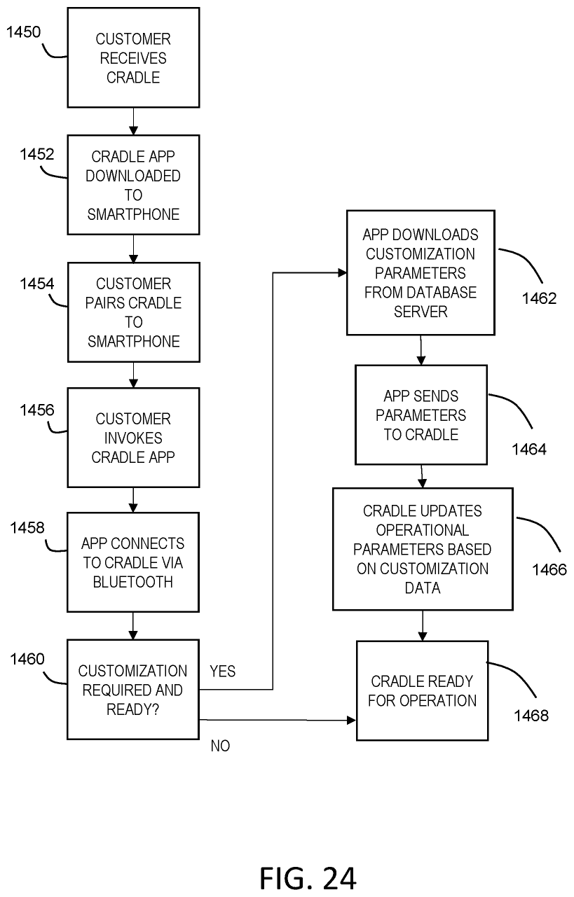

As shown in FIG. 24, the customer may pair a smartphone to the intrauterine simulation system at any point during method 1400, at block 1454. Pairing the smartphone may include downloading an app for the intrauterine simulation system to the smartphone at block 1452. The app may access the database for customization parameters 1458 that may be sent to the intrauterine simulation system at block 1464 so that the system may operate according to the customized parameters at block 1466.

H. Illustrative Combinations and Additional Examples

This section describes additional aspects and features of intrauterine simulation system, presented without limitation as a series of paragraphs, some or all of which may be alphanumerically designated for clarity and efficiency. Each of these paragraphs can be combined with one or more other paragraphs, and/or with disclosure from elsewhere in this application, including the materials incorporated by reference in the Cross-References, in any suitable manner. Some of the paragraphs below expressly refer to and further limit other paragraphs, providing without limitation examples of some of the suitable combinations.

In some examples, cradle operation is modified based on any particular at-risk cohort(s) the infant belongs to. For example, in addition to using a mother's biometric data (prepartum or postpartum) there may be circumstances where the mother's prepartum data is adjusted to account for certain infant conditions or needs. For example, a mother may collect all of her prepartum data fully and correctly, but then give birth to a pre-term baby. Being pre-term, the infant may, for example, benefit from a reduction in the motion and sound levels, e.g., due to a hypersensitivity. There may be other medical conditions of the infant that result in adjustments away from the determined/calculated initial starting cradle parameters.

Using data generated from previous at-risk infants, the infant's situation could be fine-tuned within the cohort. Using the pre-term infant as an example, other specific metrics may include: gestational age, weight, APGAR score, ethnicity, etc., and mother's age, weight, diet, drug use, etc.

A0. A baby bed, comprising:
a platform configured to support an infant;
a transducer (e.g., a low-frequency transducer) coupled to the platform;
a mechanical actuator coupled to the platform; and
an electronic controller configured to simulate an intrauterine environment for the infant by simulating a heartbeat using the transducer and simulating a walking gait using the mechanical actuator;
wherein one or more settings of the simulated intrauterine environment are determined based on biometric information of the mother of the infant.

A1. The baby bed of A0, wherein the biometric information comprises prepartum data collected from the mother of the infant.

A2.1 The baby bed of A1, wherein the biometric information comprises heartrate data.

A2.2 The baby bed of A1, wherein the biometric information comprises walking gait information.

A2.3 The baby bed of A1, wherein the biometric information is expressed in relation to time of day.

A2.4 The baby bed of A1, wherein the biometric information is expressed in relation to an activity level of the mother.

A3. The baby bed of A0, wherein the biometric information comprises postpartum data collected from the mother of the infant.

A4. The baby bed of A3, further comprising processing logic including a machine learning algorithm trained to determine the one or more settings based on the postpartum data.

A5. The baby bed of A4, wherein the electronic controller comprises the processing logic.

A6. The baby bed of A3, further comprising processing logic including a machine learning algorithm trained to infer prepartum information based on the postpartum data.

A7. The baby bed of A0, further comprising processing logic including a machine learning algorithm trained to classify a prepartum walking gait of the mother of the infant into one category of a plurality of walking gait categories, and to adjust the walking gait of the simulated intrauterine environment to match the one category.

A8. The baby bed of A0, wherein the controller is further configured to reduce a magnitude of the simulated intrauterine environment gradually over a selected timeframe.

A9. The baby bed of A0, wherein the electronic controller is in communication with one or more sensors configured to determine information relating to a real-time characteristic of the infant, and the electronic controller is configured to automatically adjust the one or more settings based on the information relating to the real-time characteristic.

A10. The baby bed of A9, wherein the real-time characteristic is a heartrate of the infant.

A11. The baby bed of A0, wherein the mechanical actuator comprises a linear-motion actuator and a rotational-motion actuator.

A12. The baby bed of A0, further comprising a mid-range transducer coupled to the platform, such that the sound waves produced by the mid-range transducer augment low-frequency waves produced by the first transducer to produce a desired spectrum of sound and/or vibration.

B0. A baby bed, comprising:
a platform configured to support an infant;
a transducer (e.g., a low-frequency transducer) coupled to the platform;
a mechanical actuator coupled to the platform; and
an electronic controller configured to simulate an intrauterine environment for the infant by simulating a heartbeat using the transducer and simulating a walking gait using the mechanical actuator;
wherein one or more settings of the simulated intrauterine environment are determined based on female biometric information.

B1. The baby bed of B0, wherein the female biometric information comprises prepartum data collected from the mother of the infant.

B2. The baby bed of B0, wherein the female biometric information comprises aggregated prepartum data collected from a plurality of mothers.

B3. The baby bed of B1, wherein the female biometric information comprises heartrate data and walking gait information in relation to time of day.

B4. The baby bed of B0, wherein the biometric information comprises postpartum data collected from the mother of the infant.

B5. The baby bed of B4, further comprising processing logic including a machine learning algorithm trained to determine the one or more settings based on the postpartum data.

B6. The baby bed of B5, wherein the electronic controller comprises the processing logic.

B7. The baby bed of B4, further comprising processing logic including a machine learning algorithm trained to infer prepartum information based on the postpartum data.

B8. The baby bed of B0, wherein the controller is further configured to reduce a magnitude of the simulated intrauterine environment gradually over a selected timeframe.

B9. The baby bed of B0, wherein the electronic controller is in communication with one or more sensors configured to determine information relating to a real-time characteristic of the infant, and the electronic controller is configured to automatically adjust the one or more settings based on the information relating to the real-time characteristic.

B10. The baby bed of B9, wherein the real-time characteristic is a heartrate of the infant.

B11. The baby bed of B0, wherein the mechanical actuator comprises a linear-motion actuator and a rotational-motion actuator.

B12. The baby bed of any one of paragraphs B0 through B11, wherein information from a plurality of mothers is used for purposes of machine learning.

B13. The baby bed of any one of paragraphs B0 through B12, wherein unique adjustment of the baby bed is based on data from the mother of the infant.

C0. A method for transitioning an infant after birth using a simulated intrauterine environment, the method comprising:
providing a simulated intrauterine environment by simulating a heartbeat using a transducer coupled to a platform configured to support an infant, and simulating a walking gait by moving the platform using a mechanical actuator; and
determining and setting one or more settings or parameters of the simulated intrauterine environment based on female biometric information (e.g., biometric information of one or more mothers).

C1. The method of C0, further comprising:
simulating intrauterine audio by playing sounds through an audio speaker mounted to the platform.

C2. The method of C0, wherein the female biometric information comprises prepartum data collected from the mother of the infant, and the method further comprises collecting the prepartum data from the mother of the infant.

C4. The method of C2, wherein the female biometric information comprises heartrate data and walking gait information in relation to time of day.

C3. The method of C0, wherein the female biometric information comprises aggregated prepartum data collected from a plurality of mothers.

C4. The method of C0, wherein the biometric information comprises postpartum data collected from the mother of the infant.

C5. The method of C4, wherein determining the one or more settings of the simulated intrauterine environment includes using a machine learning algorithm trained to determine the one or more settings based on the postpartum data.

C6. The method of C4, wherein determining the one or more settings of the simulated intrauterine environment includes using a machine learning algorithm trained to infer prepartum information based on the postpartum data.

C7. The method of C0, further comprising:
reducing a magnitude of the simulated intrauterine environment gradually over a selected timeframe.

C8. The method of C0, further comprising:
using one or more sensors to determine information relating to a real-time characteristic of the infant; and
automatically adjusting the one or more settings based on the information relating to the real-time characteristic.

C9. The method of C8, wherein the real-time characteristic is a heartrate of the infant.

C10. The method of C0, wherein the mechanical actuator comprises a linear-motion actuator and a rotational-motion actuator.

C11. The method of C5 or C6, further comprising training the machine learning model using a database including prepartum and postpartum data collected from a cohort of mothers.

D0. In some embodiments, a system for transitioning an infant from an intrauterine environment comprises captured biometric data of the infant's actual mother's prepartum biometric data and used to customize the operation of the intrauterine simulation system to more realistically mimic the infant's actual intrauterine experience; means for capturing the mother's biometric data, means for storing the data locally or in the cloud, means for applying an algorithm to the data, and means for converting it to control signals for motion and sound generation of the intrauterine simulation.

E0. In some embodiments, a system for transitioning an infant from an intrauterine environment comprises biometric data of its mother captured postpartum and transforming the data using an algorithm developed from a database of collected biometrics of mothers from before and after the birth of their babies; means for capturing the mother's biometric data, means for storing the data locally or in the cloud, means for applying an algorithm to the data, means for converting it to control signals for motion and sound generation of the intrauterine simulation system.

F0. A bassinet configured to simulate an intrauterine environment, the bassinet comprising a movable platform configured to support a floor of the bassinet, the movable platform defining a longitudinal axis; a suspension system including: at least one linear-motion actuator attached to a fixed frame disposed underneath the movable platform and configured to linearly displace the movable platform along the longitudinal axis; and at least one rotational-motion actuator configured to impart rotational motion to the movable platform; a drive system configured to drive the suspension system; and a transducer disposed underneath the movable platform and configured to produce vibrations and/or sound.

F1. The bassinet of paragraph F0, wherein the movable platform, the suspension system, and the drive system are configured to be removable from the bassinet.

F2. The bassinet of any one of paragraphs F0 through F1, wherein the drive system includes an electronic controller configured to vary the linear displacement and rotational motion based on a time of day.

F3. The bassinet of any one of paragraphs F0 through F2, wherein the at least one rotational-motion actuator of the suspension system comprises a pair of cams attached to a shaft disposed transverse to the longitudinal axis, and wherein the cams are offset from each other.

F4. The bassinet of paragraph F3, wherein the drive system is configured to drive the linear-motion actuator and the pair of cams to move the movable platform in a modified figure-eight pattern.

F5. The bassinet of paragraph F4, wherein the linear-motion actuator is driven by a first pulley and the pair of cams is driven by a second pulley, and a ratio of a cycle of the first pulley to a cycle of the second pulley is approximately 2:1.

F6. The bassinet of any one of paragraphs F0 through F5, wherein the transducer comprises a bass shaker or haptic engine.

F7. The bassinet of paragraph F6, wherein the transducer is configured to produce vibrations and/or sound within substantially an entirety of the movable platform.

G0. An infant support system comprising a platform defining a longitudinal axis; means for translating the platform along the longitudinal axis; means for rotating the platform about the longitudinal axis; and means for inducing low-frequency acoustic waves and/or vibrations in the platform.

G1. The system of paragraph G0, wherein the means for translating the platform include slide shaft driven by a motor.

G2. The system of paragraph G1, wherein the means for rotating the platform include a pair of offset cams mounted to a shaft driven by the motor.

G3. The system of any one of paragraphs G0 through G2, wherein the means for inducing low-frequency acoustic waves and/or vibrations in the platform include a bass shaker configured to produce acoustic waves predominantly in a range of approximately to 80 Hz.

G4. The system of paragraph G3, further comprising means for vibrationally isolating the tactile transducer from the means for translating the platform and from the means for rotating the platform.

H0. A bassinet configured to simulate an intrauterine environment comprising a movable platform configured to support a floor of the bassinet, the movable platform defining a longitudinal axis; a suspension system including: at least one linear-motion actuator attached to a fixed frame disposed underneath the movable platform and configured to linearly displace the movable platform along the longitudinal axis; and at least one rotational-motion actuator configured to impart rotational motion to the movable platform; a drive system configured to drive the suspension system; and a transducer disposed underneath the movable platform and configured to produce vibrations having frequency components preferably in any desired range of frequencies, such as approximately 5-100 Hz or approximately 20-80 Hz for bass components, up to 20,000 Hz or even ultrasonic frequencies in other embodiments.

J0. A system for transitioning an infant from an intrauterine environment comprising a transducer coupled to an underside of a platform and configured to transmit low-frequency sound waves and/or vibrations through the platform to an upper side of the platform opposite the underside; and a motion-control system configured to translate the platform along a longitudinal axis of the platform and to rotate the platform about the longitudinal axis.

K0. An infant support system comprising a platform defining a longitudinal axis; means for translating the platform along the longitudinal axis; means for rotating the platform about the longitudinal axis; and means for inducing low-frequency acoustic waves and/or vibrations in the platform.

L0. An infant support system or method (e.g., as outlined in any paragraph above), wherein the biometric information of the mother of the infant is collected and utilized relative to her activity level(s) and/or the time of day. Heartrate and activity level are not always in sync. For example, the heartrate can increase/decrease without a corresponding change in motion/gait.

### Advantages, Features, and Benefits

The different embodiments and examples of the intrauterine simulation system described herein provide several advantages over known solutions for simulating an intrauterine environment. For example, illustrative embodiments and examples described herein allow a simulation of a maternal heartbeat that includes generation of acoustic waves and/or vibrations at frequencies characteristic of the maternal heartbeat as experienced by a fetus inside the uterus.

Additionally, and among other benefits, illustrative embodiments and examples described herein allow an infant to experience a simulated maternal heartbeat as a substantially full-body vibration associated with conduction through bone in addition to sound waves associated with the ears.

Additionally, and among other benefits, illustrative embodiments and examples described herein allow simulation of heartbeat sensations and motion associated with the intrauterine environment to be tapered off over time, thereby facilitating an infant's transition from the intrauterine environment to the extrauterine environment.

Additionally, and among other benefits, illustrative embodiments and examples described herein allow simulation of heartbeat sensations and motion associated with the intrauterine environment to have initial conditions closely replicate those of the infant's own mother and from there be tapered off over time, thereby facilitating an infant's transition from the intrauterine environment to the extrauterine environment.

Additionally, and among other benefits, illustrative embodiments and examples described herein allow initial conditions of the simulation of heartbeat sensations and motion associated with the intrauterine environment to be automatically generated through collection of mother's prepartum biometric data, then analyzed and modified via algorithms, and sent to control the device operation.

Additionally, and among other benefits, illustrative embodiments and examples described herein when mother's prepartum biometric data is not available to allow estimated initial conditions of the simulation of heartbeat sensations and motion associated with the intrauterine environment to be automatically generated through collection of mother's postpartum biometric data, then combined with a database of other mothers prepartum and postpartum biometric data and analyzed and modified via algorithms to create an estimated prepartum biometric environment of the infant's own mother, and then sent to control the device operation.

Additionally, and among other benefits, illustrative embodiments and examples described herein allow the initial conditions of the simulation of heartbeat sensations and motion associated with the intrauterine environment to be manually selected within a limited range when detailed prepartum biometric data is not available

Additionally, and among other benefits, illustrative embodiments and examples described herein allow the initial conditions of the simulation of heartbeat sensations and motion associated with the intrauterine environment to be modified by aspects of the infant's own biometric data or medical health conditions to reduce (or increase) the stimulation levels of motion and sound to match the level required by the infant. This may be done manually by parents or attending medical personnel, or automatically based on heart rate and respiration data captured in real-time (or near real-time) from the infant.

Additionally, and among other benefits, illustrative embodiments and examples described herein allow the initial conditions of the simulation of heartbeat sensations and motion associated with the intrauterine environment to be modified in real-time by feedback from aspects of the infant's biometric data or medical health conditions.

Additionally, and among other benefits, illustrative embodiments and examples described herein allow the motion to manually restart via app or control panel in cases when the infant starts crying while the motion is stopped.

Additionally, and among other benefits, illustrative embodiments and examples described herein to allow the linear displacement and angular rotation of the mechanical system to be customized to replicate the actual step length (stride) and hip rotation of the infant's own mother as she walks.

Additionally, and among other benefits, illustrative embodiments and examples described herein the maternal intrauterine heartbeat sounds and intra-abdominal sounds generated by the cradle's sound system may be an actual recording from the infant's own mother and these sounds may be associated with activity levels from standing, sitting, and lying, and are then reproduced for time periods corresponding to the device motion of walking, sitting, and sleeping.

Additionally, and among other benefits, illustrative embodiments and examples described herein include a robust suspension system capable of withstanding a long period of use (e.g., capable of sequentially and/or serially being used with a plurality of infants), and able to be shipped a plurality of times (e.g., between a plurality of homes, hospitals, and/or distribution centers) without sustaining damage or requiring excessive care.

Additionally, and among other benefits, illustrative embodiments and examples described herein allow simulation of motion and sound patterns characteristic of those experienced by a fetus during the third trimester of gestation. In contrast, known systems typically are configured only for a general rocking pattern. Accordingly, illustrative embodiments and examples described herein simulate the intrauterine experience much more closely than known systems.

Additionally, and among other benefits, illustrative embodiments and examples described herein allow production of motion quietly and smoothly, with high safety and reliability and very low maintenance.

No known system or device can perform these functions. However, not all embodiments and examples described herein provide the same advantages or the same degree of advantage.

### CONCLUSION

The disclosure set forth above may encompass multiple distinct examples with independent utility. Although each of these has been disclosed in its preferred form(s), the specific embodiments thereof as disclosed and illustrated herein are not to be considered in a limiting sense, because numerous variations are possible. To the extent that section headings are used within this disclosure, such headings are for organizational purposes only. The subject matter of the disclosure includes all novel and nonobvious combinations and subcombinations of the various elements, features, functions, and/or properties disclosed herein. The following claims particularly point out certain combinations and subcombinations regarded as novel and nonobvious. Other combinations and subcombinations of features, functions, elements, and/or properties may be claimed in applications claiming priority from this or a related application. Such claims, whether broader, narrower, equal, or different in scope to the original claims, also are regarded as included within the subject matter of the present disclosure.

What is claimed is:

1. A baby bed, comprising:
a platform configured to support an infant;
a transducer coupled to the platform;
a mechanical actuator coupled to the platform; and
an electronic controller configured to simulate an intrauterine environment for the infant by simulating a heartbeat using the transducer and simulating a walking gait using the mechanical actuator;
wherein one or more settings of the simulated intrauterine environment are determined by a machine learning algorithm based on biometric information of the mother of the infant.

2. The baby bed of claim 1, wherein the biometric information comprises prepartum data collected from the mother of the infant.

3. The baby bed of claim 1, wherein the biometric information comprises heartrate data and walking gait information in relation to time of day.

4. The baby bed of claim 1, wherein the biometric information comprises postpartum data collected from the mother of the infant.

5. The baby bed of claim 4, wherein the machine learning algorithm is further trained to estimate prepartum information based on the postpartum data.

6. The baby bed of claim 1, wherein the machine learning algorithm is further trained to classify a prepartum walking gait of the mother of the infant into one category of a plurality of walking gait categories, and to adjust the walking gait of the simulated intrauterine environment to match the one category.

7. The baby bed of claim 1, wherein the electronic controller is in communication with one or more sensors configured to determine information relating to a real-time characteristic of the infant, and the electronic controller is configured to automatically adjust the one or more settings based on the information relating to the real-time characteristic.

8. The baby bed of claim 1, wherein the mechanical actuator comprises a linear-motion actuator and a rotational-motion actuator.

9. The baby bed of claim 1, wherein the machine learning algorithm is trained on prepartum and postpartum data collected from a plurality of mothers; and
wherein the machine learning algorithm is configured to estimate prepartum data of the mother of the infant based on the biometric information of the mother of the infant.

10. The baby bed of claim 9, wherein the biometric information comprises postpartum data collected from the mother of the infant.

11. A baby bed, comprising:
a platform configured to support an infant;
a transducer coupled to the platform;
a mechanical actuator coupled to the platform; and
an electronic controller configured to simulate an intrauterine environment for the infant by simulating a heartbeat using the transducer and simulating a walking gait using the mechanical actuator;
wherein one or more settings of the simulated intrauterine environment are determined based on female biometric information.

12. The baby bed of claim 11, wherein the female biometric information comprises prepartum data or postpartum data collected from the mother of the infant.

13. The baby bed of claim 11, wherein the female biometric information comprises aggregated prepartum data collected from a plurality of mothers.

14. The baby bed of claim 11, further comprising processing logic including a machine learning algorithm trained to estimate prepartum data of the mother of the infant based on the biometric information of the mother of the infant and determine the one or more settings based on the female biometric information and the estimated prepartum data;
wherein the machine learning algorithm is trained on prepartum and postpartum data collected from a plurality of mothers; and
wherein the female biometric information comprises postpartum data collected from the mother of the infant.

15. The baby bed of claim 11, wherein the electronic controller is in communication with one or more sensors configured to determine information relating to a real-time characteristic of the infant, and the electronic controller is configured to automatically adjust the one or more settings based on the information relating to the real-time characteristic.

16. A method for transitioning an infant after birth using a simulated intrauterine environment, the method comprising:
providing a simulated intrauterine environment by simulating a heartbeat using a transducer coupled to a platform configured to support an infant, and simulating a walking gait by moving the platform using a mechanical actuator; and
determining and automatically setting one or more parameters of the simulated intrauterine environment based on biometric information of one or more mothers.

17. The method of claim 16, further comprising:
simulating intrauterine audio by playing sounds through an audio speaker coupled to the platform.

18. The method of claim 16, wherein the biometric information comprises prepartum data collected from the mother of the infant, and the method further comprises collecting the prepartum data from the mother of the infant.

19. The method of claim 16, wherein determining the one or more parameters of the simulated intrauterine environment includes using a machine learning algorithm trained to estimate prepartum data of the one or more mothers based on the biometric information of the one or more mothers and determine the one or more parameters based on the biometric information and the estimated prepartum data; and
wherein the biometric information of the one or more mothers comprises postpartum data collected from the one or more mothers.

20. The method of claim 16, further comprising:
using one or more sensors to determine information relating to a real-time characteristic of the infant; and
automatically adjusting the one or more parameters based on the information relating to the real-time characteristic.
